(19) United States
(12) Patent Application Publication
(10) Pub. No.: US 2025/0205360 A1
(43) Pub. Date: Jun. 26, 2025

(54) FUNCTIONALIZED CORE-SHELL NANOGEL SCAVENGER FOR IMMUNE MODULATION THERAPY

(71) Applicant: The Research Foundation for the State University of New York, Albany, NY (US)

(72) Inventors: Juntao Luo, Jamesville, NY (US); Xiaotian Ji, Syracuse, NY (US); Changying Shi, Jamesville, NY (US)

(21) Appl. No.: 18/852,696

(22) PCT Filed: Mar. 30, 2023
(86) PCT No.: PCT/US23/16929

§ 371 Date: Sep. 30, 2024

Related U.S. Application Data
(60) Provisional application No. 63/325,336, filed on Mar. 30, 2022.

(51) Int. Cl. A61K 47/69; A61K 9/00; A61P 37/06; B82Y 5/00

(52) U.S. Cl. CPC A61K 47/6935; A61K 9/0024; A61P 37/06; B82Y 5/00

(57) ABSTRACT

Sepsis is a life-threatening complication of host response to infection and tissue damages. It is characterized by the uncontrolled systemic inflammatory response. Immune modulation therapy hasn't demonstrated consistent benefit in the clinic, due to the dynamic, complex, and heterogenous immune response in sepsis. Spontaneous attenuation of a broad spectrum of septic molecules and cytokines is promising for effective sepsis treatment. Embodiments disclosed herein are directed to functionalized nano-sized hydrogel, i.e., nanogel (NG), via a one-pot precipitation polymerization using biocompatible, biodegradable monomers/crosslinkers and versatile polymerizable hybrid telodendrimer (TD) nanotraps (NTs) for effective septic molecules scavenging.

### CROSS REFERENCE TO RELATED APPLICATIONS

This application claims priority to U.S. Provisional Application No. 63/325,336, filed Mar. 30, 2022. The content of this earlier filed application is hereby incorporated by reference herein in its entirety.

### STATEMENT OF GOVERNMENT SUPPORT

This invention was made with governmental support under grant no. GM130941 awarded by The National Institutes of Health. The government has certain rights in this invention.

### FIELD OF THE INVENTION

The present disclosure is in the field of drug delivery systems and organic chemistry. In embodiments, the present disclosure relates to an injectable nanogel for immune modulation therapy.

### BACKGROUND

Sepsis is a complex, life-threatening hyperinflammatory syndrome associated with organ failure and high mortality due to lack of effective treatment. Despite many efforts devoted to advance the methods used to treat sepsis, there remains an unacceptably high mortality rate.

Lipopolysaccharides (LPS), also called endotoxins, structurally and functionally essential components of the outer cell wall of Gram-negative bacteria, play an important role in sepsis pathogenesis. LPS can be recognized by immune cells as a pathogen-associated molecular pattern (PAMPs) and stimulate the host systemic immune response to secret massive proinflammatory cytokines, which may lead to organ dysfunction and failures. Approaches for endotoxin neutralization and cytokine removal present a promising approach for inflammation control in sepsis treatments.

The stem of immunogenic LPS is lipid A, a glycolipid component including a hydrophilic bis-phosphorylated diglucosamine backbone and a hydrophobic domain with six or seven acyl chains. The anionic and amphiphilic feature of lipid A is an attractive target for molecular design of LPS-binder to sequester the toxin. Similarly, cytokines and many DAMPs (i.e., the Damage-Associated Molecular Patterns) and PAMPs molecules all have the feature of charges and hydrophobic domains for tertiary structure assembly, e.g., proteins, polynucleotides, which also provide opportunity to molecular binding to scavenging. Anti-cytokine therapies and LPS-attenuating therapies via cationic amphiphilic small molecules, peptides, and antibodies, have been developed, but have yet to show efficacy in reducing the mortality of sepsis. Simultaneous attenuation of both immune stimulating molecules, e.g., LPS and overflowing signaling cytokines is critical for effective sepsis treatment.

There is a continuous need for improved pharmaceutical delivery systems and treatments for subjects in need thereof.

### SUMMARY OF THE INVENTION

Embodiments of the instant disclosure include a biomolecule-binding nanogel composition comprising a plurality of nanoparticles, each nanoparticle containing a nano-sized crosslinked hydrogel system, the nanoparticle having a spherical surface and within the system a plurality of dendritic functionalized telodendrimer containing a plurality of hydrophobic groups and hydrophilic charged binding moieties, the telodendrimer is bonded to a linear polyethylene glycol polymer (PEG) chain and polymerized in the hydrogel system by at least one double bond located between the linear polyethylene glycol polymer (PEG) chain and the dendritic functionalized telodendrimer, the linear polyethylene glycol polymer (PEG) chain extending on the surface of the nanoparticle to protect a size-exclusive nanogel network.

Embodiments of the instant disclosure include a biomolecule-binding nanogel composition wherein nano-sized crosslinked hydrogel system is polymerized from at least one hydrophilic monomer selected from the group consisting of Diethylene glycol methyl ether methacrylate, Triethylene glycol methyl ether methacrylate, Tetraethylene glycol methyl ether methacrylate, and Pentaethylene glycol methyl ether methacrylate. Embodiments of the instant disclosure include a biomolecule-binding nanogel composition, wherein nano-sized crosslinked hydrogel system is polymerized from a biodegradable crosslinking monomer having a crosslinker monomer and a crosslinker molar ratio of about 0.1% to about 20%.

Embodiments of the instant disclosure include a biomolecule-binding nanogel composition, wherein the nanoparticles range in size from about 20 nm to about 1000 nm. Embodiments of the instant disclosure include a biomolecule-binding nanogel composition, wherein polyethylene glycol polymer (PEG) chain has molecular weight from about 44 Dalton to about 40,000 Dalton. Embodiments of the instant disclosure include a biomolecule-binding nanogel composition, wherein hydrophobic group is at least one selected from the group consisting of long-chain alkanes (C₁-C₅₀), fatty acids (C₁-C₅₀), aromatic molecules, esters, halogens, nitrocompounds, anthracyclines, fluorocarbons, silicones, steroids, cholesterol, terpenoids, vitamins, and polymers, and amphiphilic groups, cholic acid, riboflavin, and chlorogenic acid. Embodiments of the instant disclosure include a biomolecule-binding nanogel composition, wherein the charged binding moiety is a negatively charged moiety, derivative, or analog of hydroxyl, carboxyl, phosphate, sulfonate, methanesulfonamide, squaric acid, sulfonamide, or oxalic acid, and/or a positively charged moiety, derivative, or analog of arginine, guanidine, amidine, primary amine, secondary amine, tertiary amine, quaternary amine, or tetrazole. Embodiments of the instant disclosure include a biomolecule-binding nanogel composition, wherein the hydrophobic binding group is heptadecanoic acid (C17) and the charged binding moiety is arginine (Arg). Embodiments of the instant disclosure include a biomolecule-binding nanogel composition, wherein the biomolecules that bind to the hydrophobic groups and hydrophilic charged binding moieties include lipopolysaccharides (LPS), small molecule drugs, peptides, proteins, and cytokines.

Embodiments of the instant disclosure include a method of treating uncontrolled systemic inflammatory response in a mammal. The method comprises administering to a mammal in need thereof a therapeutically effective amount of a biomolecule-binding nanogel composition comprising, a plurality of nanoparticles, each nanoparticle containing a nano-sized crosslinked hydrogel system, the nanoparticle having a spherical surface and within the system a plurality of dendritic functionalized telodendrimer containing a plurality of hydrophobic groups and hydrophilic charged binding moieties, the telodendrimer is bonded to a linear polyethylene glycol polymer (PEG) chain and polymerized in the hydrogel system by at least one double bond located between the linear polyethylene glycol polymer (PEG) chain and the dendritic functionalized telodendrimer, the linear polyethylene glycol polymer (PEG) chain extending on the surface of the nanoparticle to protect a size-exclusive nanogel network.

Embodiments of the instant disclosure include a biomolecule-binding hydrogel composition comprising a plurality of core-shell nanoparticle, the core-shell nanoparticle having a core and a shell, the core comprises a crosslinked hydrogel system and a plurality of functionalized telodendrimers comprising a plurality of charged and hydrophobic binding moieties, the system having a crosslinking density, the shell comprises a plurality of hydrophilic crosslinked polymers to provide a coating of the entire core, the coating having a crosslinking density that is no less than the crosslinking density of the core.

Embodiments of the instant disclosure include a biomolecule-binding hydrogel composition, wherein shell hydrophilic crosslinked telodendrimers are polymerized from at least one hydrophilic monomers selected from the group consisting of Diethylene glycol methyl ether methacrylate, Triethylene glycol methyl ether methacrylate, Tetraethylene glycol methyl ether methacrylate, Pentaethylene glycol methyl ether methacrylate, N-lsopropylacrylamide (NIPAM), acrylic acid (AA), N-Hydroxyethyl acrylamide (HEAA), Carboxybetaine methacrylate (CBMA), Serine methacrylate (SrMA), Sulfobetaine methacrylate (SBMA), 2-Methacryloyloxyethyl phosphorylcholine (MPC), and 2-(N,N-diethylamino)ethyl methacrylate (DEAMA). Embodiments of the instant disclosure include a biomolecule-binding hydrogel composition, wherein the core functionalized crosslinked telodendrimers are polymerized from at least one hydrophilic monomer selected from the group consisting of Diethylene glycol methyl ether methacrylate, Triethylene glycol methyl ether methacrylate, Tetraethylene glycol methyl ether methacrylate, Pentaethylene glycol methyl ether methacrylate. Embodiments of the instant disclosure include a biomolecule-binding hydrogel composition, wherein the core contains from about 0.1% to about 20% acrylic acid for functionalized telodendrimer conjugation. Embodiments of the instant disclosure include a biomolecule-binding hydrogel composition, wherein the binding hydrophobic moiety is at least one selected from the group consisting of long-chain alkanes (C₁-C₅₀), fatty acids (C₁-C₅₀), aromatic molecules, esters, halogens, nitrocompounds, anthracyclines, fluorocarbons, silicones, certain steroids such as cholesterol, terpenoids, vitamins, and polymers, and amphiphilic groups, cholic acid, riboflavin, and chlorogenic acid. Embodiments of the instant disclosure include a biomolecule-binding hydrogel composition, wherein the charged binding moiety is a negatively charged moiety, derivative, or analog of hydroxyl, carboxyl, phosphate, sulfonate, methanesulfonamide, squaric acid, sulfonamide, or oxalic acid, and/or a positively charged moiety, derivatives, or analog of arginine, guanidine, amidine, primary amine, secondary amine, tertiary amine, quaternary amine, or tetrazole. Embodiments of the instant disclosure include a biomolecule-binding hydrogel composition, wherein the hydrophobic moiety is heptadecanoic acid (C17) and the charged moieties are arginine (Arg) and/or spermine (Spm). Embodiments of the instant disclosure include a biomolecule-binding hydrogel composition, wherein shell hydrophilic crosslinked hydrogel are polymerized from poly(N-isopropylacrylamide-co-2-methacryloyloxyethyl phosphorylcholine). Embodiments of the instant disclosure include a biomolecule-binding hydrogel composition, wherein the core-shell nanoparticle ranges in size from about 20 nm to about 1000 nm. Embodiments of the instant disclosure include a biomolecule-binding hydrogel composition, wherein the shell coating prevents biomolecules having a molecular weight above about 50 kDa from entering the hydrogel composition and binding with the telodendrimer binding moieties.

Embodiments of the instant disclosure include a method of treating uncontrolled systemic inflammatory response in a mammal. The method comprises administering to a mammal in need thereof a therapeutically effective amount of a biomolecule-binding hydrogel composition comprising, a plurality of core-shell nanoparticle, the core-shell nanoparticle having a core and a shell, the core comprises a crosslinked hydrogel system and a plurality of functionalized telodendrimers comprising a plurality of charge and hydrophobic binding moieties, the system having a crosslinking density, the shell comprises a plurality of hydrophilic crosslinked polymers to provide a coating of the entire core, the coating having a crosslinking density that is no less than the crosslinking density of the core.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [Cl 95%] for the mean) or within ±10% of the indicated value, whichever is greater.

As used herein, the term “forming a mixture” or “forming a slurry” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated; however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

As used herein, the term biomolecules refers generally to protein, peptide, nucleic acids and lipids and other small molecules, e.g., heme.

As used herein, the term biocompatible, biodegradable monomers/crosslinkers describes, e.g., hydrophilic PEG or some other bio-synthetic polymers that are mostly biocompatible will not induce significant immune response and toxicity to a mammal. Biodegradable refer to the S—S containing or pH sensitive structure, or enzymatic labile structures that can be degraded in vivo by glutathione or acidic pH microenvironment in infection, inflammation or solid tumor or in lysosome in cells.

As used herein, the term cytokine adsorption, describes the capture of cytokine in solution, biofluid and blood.

As used herein, the term dendrons are tree-like molecular structures with multiple end groups, depending on the number of branching points, i.e., the dendron generation, and a focal point that enables coupling, e.g., to a polymer.

As used herein, the term dendritic, describes the branching projections of the telodendrimer, e.g., branching like a tree.

As used herein, the term endotoxin removal, describes scavenging endotoxin from a solution, biofluid or blood.

As used herein, the term hydrogel is a biphasic material, a mixture of porous, permeable solids and at least 10% by weight or volume of interstitial fluid composed completely or mainly by water.

As used herein, the term hydrophobic groups describes long-chain alkanes (C₁-C₅₀) and fatty acids (C₁-C₅₀), aromatic molecules, esters, halogens, nitrocompounds, anthracyclines, fluorocarbons, silicones, certain steroids such as cholesterol, terpenoids, vitamins, and polymers, and amphiphilic groups, cholic acid, riboflavin, and chlorogenic acid.

As used herein, the term immune modulation, describes modulation of immune system of a mammal by attenuate or delivery immune stimulating or immune regulating molecules.

As used herein, the term macromers are relatively high molecular weight species with a single functional polymerizable group which, although used as monomers, have high enough molecular weight or internal monomer units to be considered polymers.

As used herein, the term nanogel is a polymer-based, crosslinked hydrogel particle on the sub-micron scale. These complex networks of polymers present a unique opportunity in the field of drug delivery at the intersection of nanoparticles and hydrogel synthesis.

As used herein, the term nanotraps (NTs), describes the functionalized telodendrimers. They are comprised of multiple adjacent charged and hydrophobic moieties attached to the periphery of the dendritic structure. The NTs bind with septic molecules, e.g., LPS, DAMPs, PAMPs and cytokines.

As used herein, the term negatively charged group is a moiety or derivative or analog of hydroxyl, carboxyl, phosphate, sulfonate, methanesulfonamide, squaric acid, sulfonamide, or oxalic acid.

As used herein, the term positively charged group, when present, is chosen from moieties or derivatives or analogs of arginine, guanidine, amidine, secondary amine, tertiary amine, quaternary amine, or tetrazole.

As used herein, the term one step precipitation polymerization, describes a one-pot reaction to make nanogel with functionalized TD NT integrated covalently in nanogel.

As used herein, the term PEGylated (i.e., PEGylation), describes a biochemical modification process of bioactive molecules or interface with polyethylene glycol (PEG), which lends several desirable properties to increase the biocompatibility, stability and stealth property of the material for proteins/peptides, antibodies, and vesicles considered to be used for therapy or genetic modification of bio-interface

As used herein, the term radical precipitation polymerization, refers to radical polymerization reaction of thermosensitive polymers, which will precipitate into nanoparticles while molecular weight increase at certain temperature.

As used herein, the term sepsis treatment, describes controlling the overwhelming inflammation and infection control in sepsis, which are the causes of disease progression.

As used herein, the term telodendrimers (TDs), refers to hybrids of linear polymers covalently linked with different dendrimer generations and backbones, combining the linear/branched dendrimer polymers with the hyperbranched dendrons.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and other features of this disclosure will be more readily understood from the following detailed description of the various aspects of the disclosure taken in conjunction with the accompanying drawings that depict various embodiments of the disclosure, in which:

FIG. 1 presents a schematic illustration of the synthesis of core-shell nanogels: FIG. 1a presents structures of nanotraps; FIG. 1b Agarose gel electrophoresis profiles reveal the formation of FITC-LPS-nanotraps complex, as indicated by the lost mobility in migration; FIG. 1c Synthesis of core and core-shell nanogels with nanotraps; FIG. 1d Hydrodynamic sizes and zeta-potentials of core and core-shell nanogels measured by DLS (The data are presented as mean±SD, n=3); The morphology of FIG. 1e C(Spm2C172), FIG. 1f CS(Spm2C172), FIG. 1g C(Arg4C172), and FIG. 1h CS(Arg4C172) by TEM imaging. Scale bar: 200 nm.

FIG. 2 presents the size-exclusive property and LPS capture in nanogel: Agarose gel electrophoresis showing dose dependent efficacy for LPS capturing is presented in FIG. 2a by the core nanogels with arginine- or spermine-containing nanotraps at different density (e.g., 0.1%, 0.2%, 0.6%, and 2%); FIG. 2b present the core nanogels with nanotraps at different valency (e.g., mono- and bi-valent); FIG. 2c presents the LPS capture in the core and core-shell nanogels with bivalent nanotrap at 0.1% density in comparison with nanogel conjugated with polymyxin B (PMB) at 25:1 or 50:1 mass ratio; FIG. 2d presents selective adsorption of small and negatively charged model biomolecules, e.g., LPS or α-LA with the exclusion of BSA by size exclusive effect in nanogels with arginine-containing bivalent nanotraps: the core or core-shell nanogels were incubated with LPS/α-LA, BSA or the mixture of LPS/α-LA and BSA (1:1 molar ratio) before being loaded on agarose gel electrophoresis; FIG. 2e TNF-α and IL-6 levels in RAW 264.7 cell culture medium with overnight stimulation with LPS (50 ng mL-1), nanogel (10 μg mL-1), LPS/PMB (1:20 mass ratio) or LPS/nanogel (preincubated for overnight at mass ratios of 1:50, 1:100, 1:200), respectively. (n=3, mean±SD. Statistical significance was measured by one-way ANOVA).

FIG. 3 presents in vitro cytotoxicity characterization: FIG. 3a presents in vitro hemolytic activity of nanogels after incubation with red blood cells at different concentrations for 0.5, 4, and 24 h; FIG. 3b presents cell viability of RAW 264.7 and THP-1 cells after 72 h incubation with nanogels C(Spm2C172), CS(Spm2C172), C(Arg4C172), and CS(Arg4C172), respectively, at different concentrations. The data were represented as the mean±SD based on three separate experiments.

FIG. 4 presents reduced cell uptake of the core-shell nanogels. The fluorescent microscopic images of FIG. 4a RAW264.7 and FIG. 4b THP-1 cells after incubation with the core and core-shell nanogel encapsulated with Cy3-α-LA at a concentration of 250:5 μg mL-1 for 30 min at 37° C.: cell nucleus was stained into blue with DAPI, red color is Cy3-α-LA. Scale bar: 25 am.

FIG. 5 presents LPS attenuation via core-shell nanogels in vivo: The level of cytokines is presented in FIG. 5a TNF-α and FIG. 5b IL-6 in the plasma and peritoneal lavage fluids of BALB/c mice were analyzed at 2 h after the i.p. injection of LPS (0.1 mg kg-1), nanogel with (Arg4C172) moieties (10 mg kg-1), or nanogel/LPS (0.1 mg kg-1/10 mg kg-1, nanogels were preincubated with LPS for overnight before i.p. injection). FIG. 5c presents the numbers of white blood cells and FIG. 5d the neutrophils in these mice were analyzed 2 h after treatments. FIG. 5e presents real-time in vivo bioluminescent imaging and FIG. 5f presents the quantitative bioluminescent analysis of the HLL NF-κB reporter mice were recorded at time 0, 2, 6, 24 h after i.p. injection of PBS, CS(Arg4C172) (10 mg kg-1), LPS (0.1 mg kg-1), or LPS/CS(Arg4C172) (0.1 mg kg-1/10 mg kg-1, preincubated overnight), respectively. Bioluminescence was acquired by in vivo imaging system (IVIS) 10 min after i.p. injection of luciferin solution (200 μL/20 g mice, dose of 150 mg kg-1) (n=3-5, mean±SD. One-way ANOVA has been applied for the comparison between multiple groups).

FIG. 6 presents in situ LPS attenuation via core-shell nanogels in vivo: FIG. 6a presents real-time in vivo imaging and FIG. 6b presents the relative bioluminescent flux normalized by the background signal before treatment (t=0); and FIG. 6c presents the area under curve (AUC) of relative flux for LPS-induced peritoneal inflammation in HLL mice treated, respectively, with LPS (0.1 mg kg-1), CS(Arg4C172) (20 mg kg-1), or CS(Arg4C172)/LPS (0.1 mg kg-1/20 mg kg-1; LPS was i.p. injected 5 min after nanogel i.p. injection) at 0, 2, 6, 24 h. Basal bioluminescence level was acquired by IVIS. Luciferin solution (150 mg kg-1) was i.p. injected 10 min before the abdominal bioluminescence acquisition (the data are presented as mean±SD, n=3).

FIG. 7 presents the management of hyperinflammation with nanogels in vitro: The removal of cytokines TNF-α and IL-6 by core and core-shell nanogel with nanotraps is compare with the nanogel with PMB in FIG. 7a and FIG. 7b, RAW 264.7 cell culture medium after LPS stimulation (50 ng mL-1) for 24 h; FIG. 7c and FIG. 7d present sepsis mice plasma; and FIG. 7e and FIG. 7f present peritoneal lavage collected from mice 24 h after the CLP surgery (n=3, mean±SD; statistical significance was measured by one-way ANOVA).

FIG. 8 presents cytokine scavenging and LPS adsorption in human blood: FIG. 8a and FIG. 8b present how TNF-α and IL-6, respectively, can be effectively adsorbed by the functionalized core-shell nanogel from septic patients after overnight incubation at 0.2 mg mL-1; FIG. 8c presents endotoxin activity in stimulating neutrophil for redox production was applied to evaluate the efficiency of nanogel (0.2 μg mL-1) for attenuation of LPS doped (1 ng mL-1) in healthy human blood (n=3, mean±SD; statistical significance was measured by one-way ANOVA. *: p<0.05; **: p<0.01; ***: p<0.001; ****: p<0.0001).

FIG. 9a presents Schematic illustration for the synthesis of nanogel with telodendrimer (NG-TD); FIG. 9b schematic representation of selective LPS and cytokine removal by NG-TD; FIG. 9c hydrodynamic sizes and FIG. 9d the morphology of nanogel with 10% PEG^{5k}C17₄Arg₄ measured by DLS and TEM; FIG. 9e pictures of nanogel solution before (left) and after (right) incubated with 10 mM TCEP for 48 h; FIG. 9f the size of the nanogels (NP-BAC) without or with 10 mM TCEP; TEM micrographs of NP-BAC nanogel after FIG. 9g 24 h and FIG. 9h 48 h incubation with 10 mM DTT (the data are presented as mean±SD, n=3; TEM scale bar: 200 nm).

FIG. 10 presents the removal of (in FIG. 10a) FITC-LPS and RB-α-LA and FIG. 10b pro-inflammatory cytokines (TNF-α, IL-6 and IL-1p) by NG-TD (10% PEG^{5k}C17₄Arg₄) after incubation at different mass ratio at 37° C. for overnight; FIG. 10c Agarose gel electrophoresis profiles of the NG-TD for FITC-LPS loading in 50% FBS solution at the 25/1 and 50/1 mass ratio of nanogel to LPS and FIG. 10d in the presence of excess BSA at 50/1 mass ratio of nanogel to LPS; cytokines level of FIG. 10e TNF-α and FIG. 10f IL-6 in RAW 264.7 cell culture medium with overnight stimulation with LPS (50 ng/mL), LPS/PMB (1:20 mass ratio) or LPS/NG-TD (10% or 15% PEG^{5k}C17₄Arg₄) (preincubated for overnight or added separately at mass ratios of 1:50, 1:100, 1:200), respectively. (n=3, mean±SD).

FIG. 11 presents the management of hyperinflammation with NG-TD in vitro. The removal of cytokines TNF-α and IL-6 by NG-TD in compare with the TD in FIG. 11a RAW 264.7 cell culture medium after LPS stimulation (50 ng mL-1) for 24 h; FIG. 11b human patient plasma; FIG. 11c sepsis mice plasma and FIG. 11b peritoneal lavage collected from mice 24 h after the CLP surgery (n=3, mean±SD).

FIG. 12a presents cell viability assays on RAW264.7, HFF-1 and THP-1 cells after 72 h incubation with the NG-TD as measured by MTS assay; FIG. 12b presents hemolytic property at 24 h after the red blood cell suspension was mixed with the NG-TD at different concentrations; FIG. 12c presents the fluorescent microscopic images of RAW264.7 cells after incubation with the PEI, TD, NG-TD encapsulated FITC-α-LA at a concentration of 5 μg mL⁻¹ of α-LA for 30 min at 37° C.: cell nucleus was stained into blue with DAPI, green color is FITC-α-LA. Scale bar: 25 μm.

FIG. 13a presents plasma concentration-time profile of NG-TD (50 mg/kg) and TD after administration as a single dose i.v. injection. AUC (mg-h/L): area under the concentration time curve; T_{1/2} (h): half-life; data are expressed as the mean±SD (n=3); FIG. 13b presents the concentration of TD determined by fluorescence in organs 28 h after i.v. injection.

FIG. 14a presents experimental procedures for the LPS induced infection model with wild type mice. NG (50 mg/kg) or PBS were i.v. injected into mice 5 min after i.v. injection of LPS (0.1 mg/kg). Blood was collected from tail vain at 2 and 4 h after LPS injection for ELISA test; FIG. 14b presents key cytokines TNF-α and IL-6 in plasma of PBS and NG treated groups compare with normal mice (n=3-4, mean±SD);

FIG. 14c presents experimental procedures for the LPS induced infection model with HIV-LTR/Luciferase (HLL) NF-κB reporter mice. NG (50 mg/kg) or PBS were i.v. injected into mice 5 min after i.v. injection of LPS (0.1 mg/kg). Bioluminescence was acquired by in vivo imaging system (IVIS) 10 min after i.p. injection of luciferin solution (150 mg/kg) at t=0, 2, 4, 6, 24, 48 h; (d) the quantitative bioluminescent analysis and FIG. 14e presents real-time in vivo bioluminescent imaging of the HLL mice were recorded different time point (n=3, mean±SD).

FIG. 15a presents a schematic illustration of sepsis mouse model induced by CLP surgery, and the treatment with NG-TD (10% PEG^{5k}C17₄Arg₄). The mice were administrated with NG (50 mg/kg) at 1 h, day 2, day 3, day 4 and day 5 post CLP by i.v. injection; FIG. 15b presents the survival of the CLP mice (n=8-9) with or without treatment with NG-TD monitored in 28 days; FIG. 15c presents the cytokines of (a) TNF-α and (b) IL-6 in plasma collected through tail vain 24 h post CLP; FIG. 15e body weight and FIG. 15f body temperature of the survived CLP mice in 28 days.

FIG. 16a presents Tissue histology stained with hematoxylin and eosin for major organs of septic mice in different treatment groups. Serum levels of the kidney function indicators blood FIG. 16b creatinine and FIG. 16c urea nitrogen (BUN) and FIG. 16d aspartate transaminase (AST) in mice of different treatment groups.

FIG. S1 presents synthetic route for solid-phase peptide synthesis (SPPS) of nanotraps Arg₄C17₂ and Spm2C17₂.

FIG. S2 presents MALFI-TOF MS spectra of nanotraps Arg₄C17₂ and Spm2C17₂ and intermediate compounds cleaved from Rink resin.

FIG. S3 presents H NMR spectra of nanotraps Arg₄C17₂ and Spm2C17₂ in DMSO-d₆.

FIG. S4 presents the morphology of blank (a) core and (b) core-shell nanogel by TEM images. Scale bar: 0.2 μm.

FIG. S5 presents hydrodynamic sizes of core and core-shell nanogels with 0.1% AAc before and after nanotraps conjugation measured by DLS. (mean±SD, n=3)

FIG. S6 presents hydrodynamic sizes of core nanogels with 2%, 0.6% and 0.2% AAc before and after Arg₄C17₂ nanotrap conjugation measured by DLS. (mean±SD, n=3)

FIG. S7 presents agarose gel electrophoresis profiles of the blank core and core shell nanogels for the RB-BSA, FITC-α-LA and FITC-LPS loading at the 100/1 mass ratio of nanogel to protein.

FIG. S8 presents fluorescent polarization monitoring LPS complexation with nanogel or PMB. FITC-LPS form complex with nanogels in PBS monitored by the increased fluorescent polarization.

FIG. S9 presents agarose gel electrophoresis profiles reveal the complex formation of the FITC-labeled LPS with nanogel, PMB form less-stable complex with LPS in electrophoresis, which was also unable to dissociate LPS-nanogel nanocomplex with 4-fold excess in mass ratio.

FIG. S10 presents cell viability of HFF-1, RAW 264.7 and HTP-1 cells after 72 h incubation with blank core and core-shell nanogels at different concentrations. The data was represented as the mean±SD, n=3.

FIG. S11 presents agarose gel electrophoresis profiles of Cy3-α-LA loading by nanogels C(Spm2C172), CS(Spm2C172), C(Arg₄C17₂) and CS(Arg₄C17₂) at nanogel/α-LA mass ratio of 50:1.

FIG. S12 presents quantitative result of fluorescent intensity of (a, b) RAW264.7 and (c, d) THP-1 cells after incubation with the core and core-shell nanogel encapsulated with Cy3-α-LA at a concentration of 250:5 μg/mL for 0.5 h at 37° C. The data was represented as the mean±SD, n=3. Statistical significance was measured by one-way ANOVA.

FIG. S13 presents human and mice cytokines TNF-α and IL-6 level after 12 h incubation with nanogels CS(Arg₄C17₂) at different ratio (n=3, mean±SD. Statistical significance was measured by one-way ANOVA).

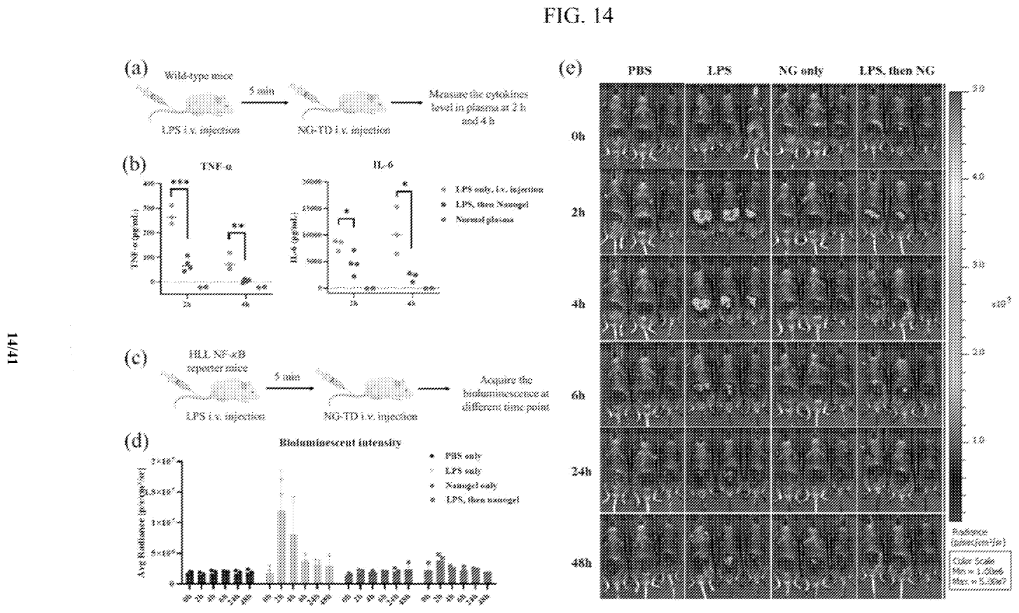

FIG. S14 presents FIG. 14a depicting the synthesis rout of N, N′-Bis (acryloyl) cystamine (BAC); FIG. 14b presents ¹H NMR, FIG. 14c presents ¹³C NMR spectra in DMSO-d₆ and FIG. 14d presents Mass spectra of BAC.

FIG. S15 presents the cumulative TD (PEG^{5k}RB₂) release from nanogel with different formulations in PBS.

FIG. S16 presents the hydrodynamic sizes of nanogels with different telodendrimers measured by DLS.

FIG. S17 presents FIG. S17a depicting a graphic illustration hydrodynamic sizes of nanogel with undegradable crosslinker (NG-BIS) with or without incubation with 10 mM TCEP at different time point; the morphology of FIG. S17b NG-BIS and FIG. S17c NG-BIS incubated with 10 mM TCEP for 72 h by TEM imaging; FIG. S17d hydrodynamic sizes of nanogel with degradable crosslinker (NG-BAC) with or without incubation with 10 mM GSH at different time point; the morphology of NG-BAC incubated with 10 mM GSH for FIG. S17e 8 h and FIG. S17f 24 h by TEM imaging. Scale bar: 200 nm.

FIG. S18 presents agarose gel electrophoresis profiles of FIG. S18a TD with different charge groups and FIG. S18b blank nanogel for the FITC-LPS loading at different mass ratio of nanogel/TD to LPS.

FIG. S19 presents agarose gel electrophoresis profiles of the nanogel with different ratio of TD (PEG^{5k}-C17₄Arg₄, 5%-20%) for the FITC-LPS loading at different mass ratio of nanogel to LPS.

FIG. S20 presents agarose gel electrophoresis profiles of the nanogel with TD (PEG^{5k}-OA₄Arg₄ or PEG^{5k}-COOH₄Arg₄) for the FITC-LPS loading at different mass ratio of nanogel to LPS.

FIG. S21 presents agarose gel electrophoresis profiles of the NG-TD for RB-α-LA loading in 50% FBS solution at the 25/1 and 50/1 mass ratio of nanogel to α-LA.

FIG. S22 presents the removal of cytokines TNF-α and IL-6 by NG with negatively charged TD RAW 264.7 cell culture medium after LPS stimulation (50 ng mL-1) for 24 h.

FIG. S23 presents the agarose gel electrophoresis profiles of FITC-α-LA loading by nanogels, TD, and PE in FIG. S23al; and FIG. S23b presents quantitative result of fluorescent intensity of RAW264.7 cells after incubation with PEI, TD, NG-TD (10%) and NG-TD (15%) encapsulated FITC-α-LA at a concentration of 5 μg/L of α-LA for 30 min at 37° C. The data was represented as the mean±SD, n=3.

FIG. S24 presents the TEM images of NG/LPS complex in FIG. S24a PBS and FIG. S24b 50% mice plasma.

FIG. S25 presents a schematic representation and hydrodynamic sizes of (FIGS. S25a and S25c) TD (PEG^{5k}-RB₂) and (FIGS. S25b and FIG. S25d) NG-TD (PEG^{5k}-RB₂).

FIG. S26 presents in FIG. S26a an experimental procedures for the LPS induced infection model with wild type mice. NG (50 mg/kg) or PBS were i.v. injected into mice, 5 min later LPS was injected (0.1 mg/kg); FIG. S26b presents key cytokines TNF-α and IL-6 in plasma of PBS and NG treated groups compare with normal mice (n=3-4, mean±SD); FIG. S26c presents experimental procedures for the LPS induced infection model with HIV-LTR/Luciferase (HLL) NF-κB reporter mice. LPS (0.1 mg/kg) was i.v. injected into mice 5 min after NG (50 mg/kg) or PBS i.v. injection of; FIG. S26d presents the quantitative bioluminescent analysis and FIG. S26e presents real-time in vivo bioluminescent imaging of the HLL mice were recorded different time point (n=3, mean SD).

FIG. S27 presents a schematic of nanogel with telodendrimer (NG-TD) formation utilizing various crosslinkers with TEGMMA (Triethylene glycol methyl ether methacrylate).

FIG. S28 presents the names and structures of monomers disclosed in embodiments herein.

FIG. S29 presents the names and structures of cross linker disclosed in embodiments herein.

### DETAILED DESCRIPTION OF THE INVENTION

Sepsis is a life-threatening complication of bacterial infection characterized by uncontrolled systemic inflammatory response. Despite many efforts devoted to finding an effective treatment, the mortality rate in sepsis is very high, and the number of hospitalizations resulting from the condition continues to rise. Lipopolysaccharide (LPS) or endotoxin is a major constituent of the outer cell wall of Gram-negative bacteria and is strongest immune stimulating compounds in nature. Upon entering the blood circulation, it may induce systemic inflammation and sepsis, which has a fatal outcome in many cases. Emerging evidence suggests that the systemic spread of endotoxin from sites of infection, rather than bacteremia itself, is crucial in the pathogenesis of this dramatic immune dysregulation. LPS interacts with a variety of plasma proteins and activates various immune competent cells. On activation, these cells produce potent pro-inflammatory mediators, e.g., tumor necrosis factor (TNF), interleukin-1 (IL-1), nitric oxide (NO), IL-6, and IL-8. During sepsis, the release of PAMPs and DAMPs perpetuates systemic inflammatory response, which result in overexpression of cytokines in serum and tissues and lead to multiple-organ failure. Therefore, timely intervention to reducing systemic circulating endotoxin and inflammatory cytokines is critical for managing sepsis.

LPS consists of a hydrophobic part, the membrane-anchored lipid A moiety, having a bisphosphoryl diglucosamine backbone to which up to seven acyl chains are ester- or amide-linked. The anionic and amphiphilic feature of lipid A is an attractive target for molecular design of LPS-binder to sequester the toxin. Interestingly, most proinflammatory cytokines are negatively charged proteins and have small to medium molecular weights (less than 30 kDa).

Embodiments disclosed herein are directed to a core-shell hydrogel nanoparticle having the core functionalized with telodendrimers (TD) nanotraps (NT), which exhibit high binding affinity to LPS and pro-inflammatory cytokines utilizing multiple charge-hydrophobic moieties and size selectivity to control hyperinflammation in sepsis. The telodendrimers are hybrids of linear polymers covalently linked with different dendrimer generations and backbones. The telodendrimers provide architectural complexity to the polymer structure. The nanotraps are a part of the functionalized telodendrimers that make up the core of the core-shell nanoparticle.

The nanotraps are multiple-charge hydrophobic moieties that bind with septic molecules, e.g., LPS and proinflammatory cytokines. The core telodendrimers can be functionalized with ligands that can be any small molecule, nucleotides, peptide or protein for tissue specific targeting. The hydrophobic moieties contain amine groups that are conjugated, for example, by condensation to the carboxylic group of AAc present in the poly(NIPAm-co-AAc-co-MPC) core to provide stability. The combination of multivalent charged and hydrophobic moieties in TD enables effective binding with biomolecules in NT.

Embodiments of the present disclosure include higher crosslinking in the shell structure of nanogel, which excludes the abundant large serum proteins and allows for size-selectivity in scavenging the medium-sized septic molecules (10-30 kDa), e.g., lipopolysaccharides, thus reducing cytokine production. At the same time, the core-shell TD NT nanogel captures the overflowing proinflammatory cytokines effectively both in vitro and in vivo from biological fluids to further control hyperinflammation. Embodiments of the present disclosure include intraperitoneal injection of the core-shell TD NT nanogel, which effectively attenuates NF-κB activation and cytokine production in LPS-induced septic mouse models. These results indicate the potential applications of the injectable TD NT core-shell nanogel to attenuate local or systemic inflammation.

Embodiments of the present disclosure include a size exclusive hydrogel functionalized with telodendrimer (TD) nanotrap (NT), (TD NT) hydrogel resin that has demonstrated 100% survival when combined with antibiotics in a severe sepsis mouse model and can be readily incorporated into the standard clinical hemoperfusion treatment for sepsis. Embodiments of the present disclosure include an injectable core-shell TD NT hydrogel nanoparticle with a size that is not limited and ranging from about 20 to about 1000 nm that provides for a selective septic molecule binding property.

Embodiments of the present disclosure include intraperitoneal injection the subject core-shell nanogel that provide effective attenuation of NF-κB activation and cytokine production in LPS-induced septic mouse models.

Embodiments of the present disclosure include a core-shell TD NT hydrogel utilizing a polyethylene glycol (PEG) shell on the surface of the particles, which can inhibit the unspecific binding. The size-exclusive nanogel network can exclude the unwanted abundant large serum proteins. According to embodiments of the present disclosure the nanogel can be formulated by one step precipitation polymerization. The nanogels have low toxicity and effectively sequester bacterial endotoxin and proinflammatory cytokines both in vitro and in vivo. In addition, the nanogel can significantly improve the survival in CLP induced sepsis mice, which is promising to serve as a novel injectable therapeutic modality to attenuate hyperinflammation for bacteremia sepsis treatment.

Embodiments disclosed herein are directed to functionalized nano-sized hydrogel, i.e., nanogel (NG), via a one-pot precipitation polymerization using biocompatible, biodegradable monomers/crosslinkers and versatile polymerizable hybrid telodendrimer (TD) nanotraps (NTs) for effective septic molecules scavenging. The TD NG hydrogel has spherical morphology with homogenous size distribution of about 300 nm, but is not limited. TD NG hydrogel is stable in plasma and can be degraded in the presence of reducing agents, e.g., TCEP and glutathione. The size-exclusive nanogel network and the shell coating, e.g., polyethylene glycol (PEG), exclude the abundant serum proteins for selective and effective capture of septic molecules, e.g., LPS, TNF-α, IL-1β, IL-6, etc., in cell culture medium, septic mouse plasma and peritoneal lavage and septic patient plasma. PEGylated TD NG is nontoxic in cell culture, low immune cell uptake, biocompatible after i.v. injection with prolonged circulation time and can be excreted into feces via liver-bile ducts. Systemic administration of TD NG significantly inhibits the LPS-induced inflammation revealed by the bioluminescent imaging, indicating NF-κB activation in HLL mice. PEGylated TD NG can effectively attenuate inflammatory molecules in situ for effective immune modulation, which ameliorate tissue damage and improve the survival rate in severe CLP sepsis mouse models.

Embodiments disclosed herein are directed to functionalized nano-sized hydrogel, wherein the shell of the core-shell nanoparticle is a hydrophilic layer of polymer, e.g., PEG, or any suitable crosslinked hydrophilic polymer, as known in the art, and the core of the core-shell nanoparticle is crosslinked hydrogel network. The telodendrimers are functionalized with moieties conjugated in the core of nanogel. Embodiments disclosed herein include telodendrimers that can be defined as, e.g., D(L-R)x, wherein subscript x is an integer from 2 to 40, D is a dendritic structure, L is linker, and R is peripheral group conjugated on the dendron with hydrophobic, or charged groups or the combination of both.

Embodiments disclosed herein are directed to biomolecule-binding functionalized nano-sized hydrogel that can be applied for both scavenging and/or therapeutic delivery of proinflammatory molecules, proteins, lipids, carbohydrates, nucleic acids, or for therapeutic delivery, e.g., small molecular drug, peptide or protein, and the like.

Interestingly, most proinflammatory cytokines are negatively charged proteins and have smaller to medium molecular weights (less than 30 kDa) compared to the abundant serum proteins. A size exclusive hydrogel resin functionalized with telodendrimer nanotrap (TD NT) was developed, which exhibits high binding affinity to LPS and pro-inflammatory cytokines with both charge and size selectivity. TD NT hydrogel resin has demonstrated a cure when combined with antibiotics in a severe sepsis mouse model. This TD NT hydrogel resins with size 200-500 um are readily incorporated into the standard clinical hemoperfusion therapy for sepsis treatment. Alternatively, the development of small-sized injectable nanoparticles with the similar selectivity and affinity to the broad spectrum of septic molecules is promising to attenuate systemic and local inflammation in situ for easy clinical administration and possible for better efficacy in sepsis treatment.

Core-shell nanogels have been extensively studied for biomedical applications with the engineerable particle properties and functionality. Different from solid nanoparticles, the swelling hydrogel structure in nanogel allows drug molecules or proteins to diffuse in and out for controlled drug release. In addition, the core can be functionalized with a ligand to capture specific analytes for biomedical detection. In embodiments, the shell with higher density on the surface can prevent large proteins, such as plasma proteins, from entering into the core. In addition, the biocompatible shell can be designed to shield the core and payload for in vivo applications. As such, the structure and density of the shell can be controlled to alter porosity and permeability. Using precipitation polymerization, the parameters including size, crosslinking density and the incorporation of functional groups in both core and shell compartments can be precisely controlled.

In embodiments telodendrimers are suitable for use with or disposed within the nanogels of the present disclosure. Non-limiting examples of suitable telodendrimers include those described in U.S. Patent Publication Nos.: 20140363371, 20150056139, 20170252456, 20170266292, 20170290921, 20190292328, 20190328742, 20200009069, 20200254012, 20210269601, and 20210317234 (all of which are entirely incorporated by reference). In embodiments, any suitable telodendrimer can be immobilized in a nanogel embodiment of the present disclosure for different therapeutic delivery, such as for small molecular drugs, protein or gene molecules for controlled release, or applied as empty for scavenging of immune stimulating, regulating molecules, toxin etc.

In embodiments, the present disclosure includes a biodegradable injectable size-exclusive nanogel (˜200 nm) to immobilize telodendrimer nanotrap for systemic immune modulation therapy by scavenging inflammatory molecules. In embodiments, the biodegradable nanogel nanotrap can be injected into patient blood and distributed into tissue for effective and quick inflammation control in critical illness, e.g. sepsis.

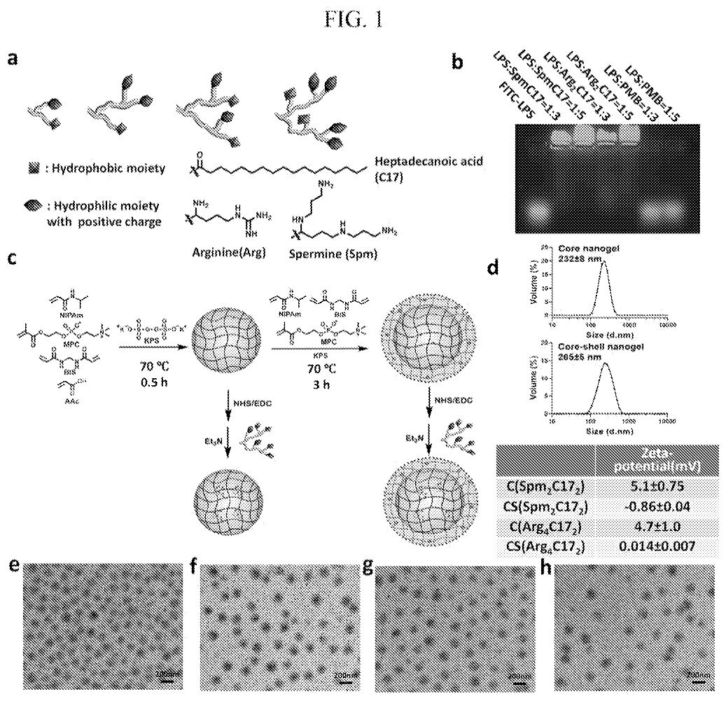

Synthesis of Nanotraps: Negatively charged biomolecules such as LPS and proinflammatory cytokines can be effectively captured by telodendrimers (TD) comprising positive charges and hydrophobic moieties. Here oligomers were designed with multiple hydrophobic heptadecanoic acid (C17) moieties and positive charged moieties, such as arginine (Arg) or spermine (Spm) on the periphery of TD for efficient binding with the negatively charged proteins and LPS (FIG. 1a). C17 can fit into diverse hydrophobic domain in protein with the flexible chain. Positively charged groups on the oligomers can interact with the phosphate groups on LPS. The oligomers were synthesized by solid phase peptide synthesis (SPPS), see description in FIG. S1. Intermediates in the synthetic process can be cleaved from the resin for the structural characterization by MALDI-TOF MS (FIG. S2). The chemical composition of the final oligomers Arg₄C17₂ and Spm2C17₂ were confirmed by ¹H NMR (FIG. S3). The oligomers show high binding affinity with LPS isolated from E. coli (FIG. 1b). In contrast, the PMB-LPS complex has moderate micromolar binding affinity and dissociates in electrophoresis.

Hydrogel Nanoparticle Synthesis and Characterization: According to embodiments of the instant disclosure, the core and core-shell nanogels were prepared by precipitation polymerization in aqueous solution with BIS as crosslinker (FIG. 1c). NIPAm-based particles were chosen because of its unique thermal-sensitive property, as the monomer is readily soluble under the reaction conditions, while the newly formed polymers undergo a phase separation and aggregate into dense polymer globules during polymerization at high temperature. Meanwhile, it is feasible to control the particle size and the effective porosity by changing the percentage of cross-linking agent and temperature. Sodium dodecyl sulfate (SDS) is the surfactant used in the nanogel syntheses, which can stabilize the growing nuclei against aggregation early in the reaction. Zwitterionic monomer MPC was also applied to increase the hydrophilicity of the nanogel. AAc (0.01%-2%) with carboxyl acid group was incorporated into the nanogel as anchors to introduce the nanotrap into the particles. During the shell coating step, the core particles serve as nuclei to graft/adhere the newly polymerized polymer chains. The permeability of the shell can be altered by adjusting the thickness and the crosslinking degree of the shell. A core was synthesized with low crosslinking degree 2% and size around 230 nm by Dynamic Light Scattering (DLS) in solution. After covering the core particle with the shell, the size of the nanogel increase around 30 nm (FIG. 1d). The higher crosslinking degree (5%) was applied in the shell structure to establish the size exclusive effects. The nanotrap was immobilized by conjugating amino groups in the oligomers to the NHS/EDC pre-activated carboxylic groups in the core particles (FIG. 1c). The introduction of the nanotrap did not change the sizes of the nanogel (FIG. S4) due to low percentage of AAC introduced to ensure efficient molecular diffusion. The morphology of the nanogel was revealed under TEM imaging (FIG. 1e-h and FIG. S6). The conjugation of nanotraps did not change the morphology of the nanogel. The covering of shell can increase the size of the nanogel from 150±15 nm (Core(Spm₂C17₂)) and 147±12 nm (Core(Arg₄C17₂)) to 173±14 nm (Core-Shell(Spm₂C17₂)) and 162±11 nm (Core-Shell(Arg₄C17₂)). Although the core-shell (CS) structures were barely recognizable under TEM imaging due to the similar chemical components, the zeta potential of nanogel significantly decreases to neutral after shell (S) coating on the positively charged core particles with nanotrap conjugated (FIG. 1d).

Optimization of the properties of core-shell nanogel: In a core-shell architecture, the properties of the core and shell can be tailored separately for particular application. Embodiments disclosed herein include a nanogel core that is effective for binding small sized cytokines and LPS; thus, a loose network in the core (2% crosslinking degree) is favorable for analytes diffusion and capture. A shell with higher crosslinking degree (5%) was separately coated onto the core, which has a denser network than the core to create size-exclusive effects to exclude the essential large serum proteins.

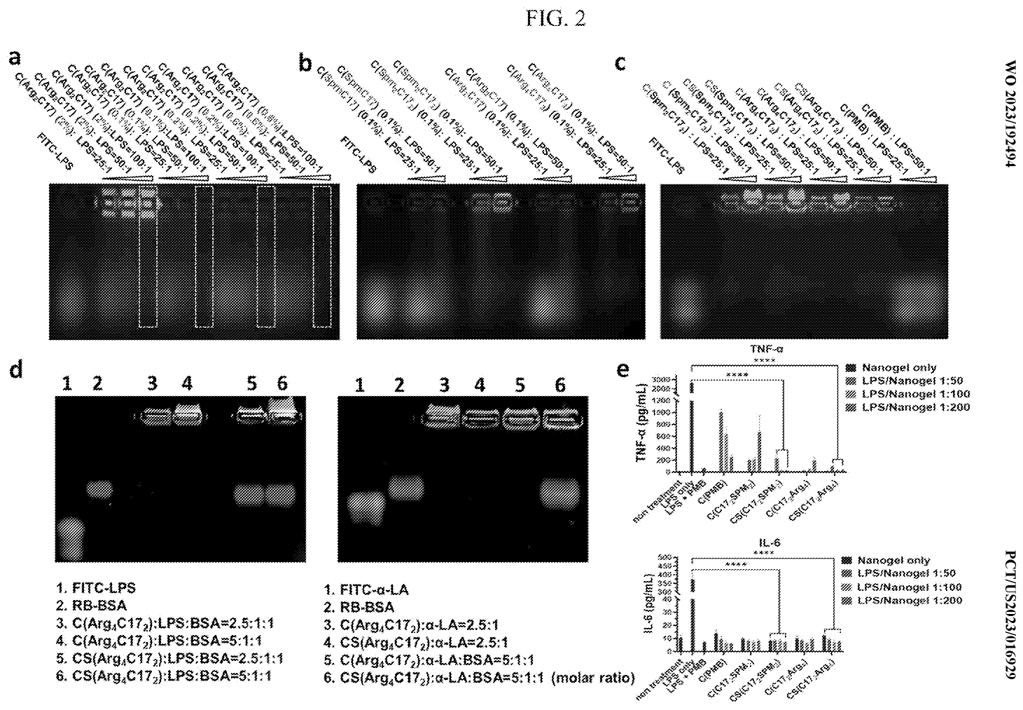

The loading capacity of the core nanogel can be optimized by both density and valency of the nanotraps. The density can be adjusted by the percentage of AAc in the core nanogel for nanotrap conjugation via amide bond formation. The nanogel was first prepared with a AAc density of 2% for nanotrap immobilization. As shown on agarose gel, fluorescently labeled LPS can be completely encapsulated into the nanogel at LPS/particle mass ratio of 100/1 (FIG. 2a). However, the reduced particle sizes of nanogel from 295 nm to 260 nm was observed (FIG. S6) after nanotraps conjugation at 2% density. This is due to the aggregation of nanotraps at high density resulting in the contraction of hydrogel network, which may hinder the kinetics for analytes to penetrate and capture. Therefore, a series of nanogel with reduced AAc density of 0.1%, 0.2% and 0.6%, in the core respectively was prepared. As expected, less impact on particle sizes was observed after nanotraps conjugation at the reduced density (FIGS. S5 and S6). More importantly, the reduced density did not sacrifice the capacity of nanogel for LPS capture (FIG. 2a). All the core nanogel with mono-valent nanotrap (Arg₂C17 and SpmC17) at density from 0.1% to 2% can completely absorb LPS at 100/1 mass ratio, which corresponding to the molar ratios of nanotrap to LPS ranging from 10:1 to 200:1. The higher nanotraps density in nanogel will self-assemble into nano-domain similar to micelles formed by telodendrimers in solution, which may not be accessible for LPS or protein binding due to the restricted mobility in hydrogel network, thus resulting in low LPS binding capacity even at high 2% AAc density. The fluorescent signal of FITC-LPS was enhanced significantly after loading in the nanogel with high density of nanotraps, due to the compact hydrophobic environments, which was consistent with the nanotrap with the increased number of C17 (FIG. 2b). According to an embodiment disclosed herein, improved LPS capturing efficiency is obtained by increased valency of the nanotrap. The doubling of valence of both charge and hydrophobic moieties Arg₄C17₂ in nanotraps significantly increases the loading capacity of LPS in the core of nanogel from 100/1 to 25/1 (nanogel/LPS mass ratio) as compared to the monomeric Arg₂C17 (FIG. 2b). Similarly, the increased valency of nanotraps with spermine as the positive charges also captures LPS more effectively at a 25:1 mass ratio and 0.1% nanotrap density.

After core optimization for effective LPS adsorption, it is important to create selectivity for analytes capture from bio-fluids. A biocompatible and size-exclusive shell layer on the functional core may exclude various proteins, e.g., serum albumin and antibodies. A similar chemical component poly(NIPAm-co-MPC) except for AAc was polymerized on the surface of core nanogel via precipitation polymerization. Meanwhile, 25% MPC were incorporated to increase the hydrophilicity of surface chemistry and reduce nonspecific binding. Additionally, the crosslinking degree was increased to 5% in order to create size exclusive effects. The coverage of a shell layer did not influence the adsorption of small proteins in the core. As shown in FIG. 2c, the core-shell nanogels with the optimized density (0.1%) and valency (bivalent) of nanotrap in the core exhibited same loading capacity for LPS at 25:1 mass ratio.

Polymyxin B (PMB) is an LPS-binding antibiotic, which was also conjugated in the core of nanogel through the same procedure for LPS binding. As a comparison, it showed low binding affinity to LPS as indicated by fluorescent polarization (FP) spectrometry studies with minimum noticeable changes in FP reading (FIG. S7). Accordingly, PMB containing nanogel is also not effective for LPS capture at 50/1 mass ratio as shown in FIG. 2c. FITC-LPS incubated with the core or core-shell nanogels containing Arg₄C17₂ or Spm2C17₂ in the presence of four-times more PMB in mass relative to nanogel for competing LPS binding. As a result, LPS can be captured effectively in both core and core-shell nanogels as shown in agarose gel electrophoresis assay (FIG. S8), confirming for effective LPS binding in nanogel.

To determine the size-selective effect of core-shell nanogels, a mixture of FITC labeled LPS or α-LA and Rhodamine B labeled BSA (1:1 molar ratio) was incubated with either core or core-shell nanogels for 2 h and characterized in agarose electrophoresis (FIG. 2d). The core nanogels effectively bind to both larger BSA (67 kDa) and smaller protein (α-LA) and LPS. In contrast, the binding of larger BSA were significantly decreased after incubation with core-shell nanogels, where LPS and α-LA can still be effectively captured (FIG. 2b). This result indicates that the coverage of a higher crosslinking shell on the core particles can effectively create a size-exclusive effect to exclude large proteins, such as albumin, but remains effective for the binding of broad range of small negatively charged proteins and endotoxin.

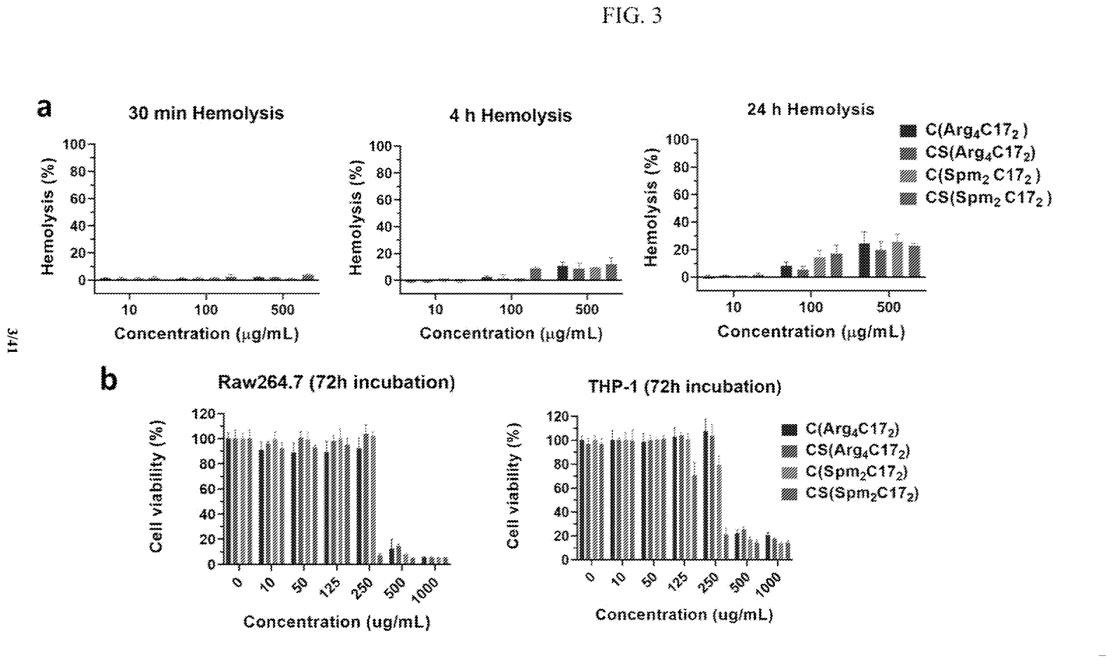

Biocompatibility and cell uptake: NIPAm containing particles have been extensively studied for biotechnological applications with respect to their thermo-responsivity. Both core and core-shell nanogels exhibit good hemocompatibility when incubated with red blood cell for 0.5 and 4 h at a concentration up to 500 μg/mL without significant hemolytic activities observed (FIG. 3a). Blank core and core-shell nanogel nanoparticles have been tested to be non-toxic up to 1 mg/mL in cell cultures with both immune cells and normal fibroblast cells (FIG. S9). After TD conjugation, nanogels still exhibit good biocompatibility without any toxicity up to 250 μg/mL in cell culture with immune cells (FIG. 3b), e.g. RAW264.7 (murine macrophage) HTP-1 (human monocyte). Interestingly, the core and core-shell nanogel with nanotrap Arg₄C17₂ show better biocompatibility than nanogel with Spm2C17₂. The cells show 100% survival at concentration at 250 μg/mL after 72 h incubation with C(Arg₄C17₂) and CS(Arg₄C17₂).

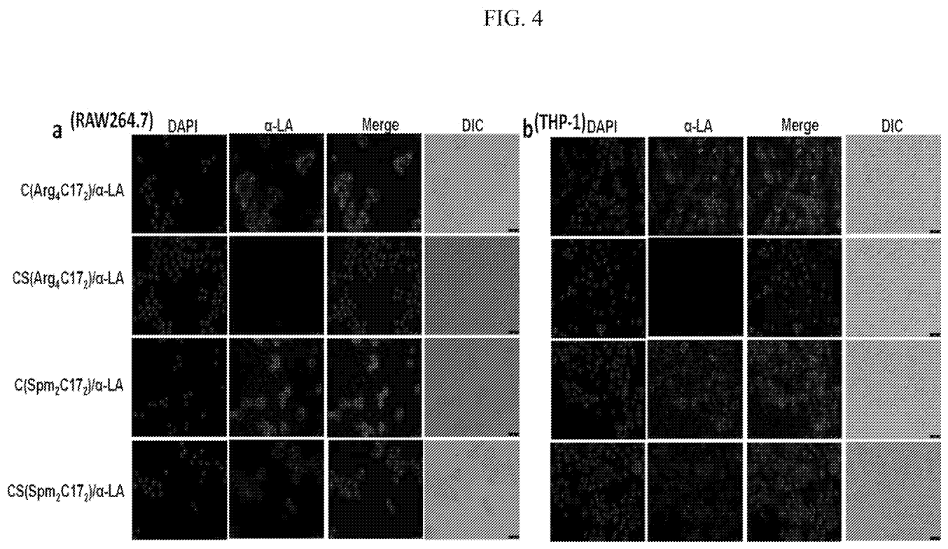

The nanotrap is designed to scavenge circulating septic molecules and cytokines, the phagocytosis of nanogel is preferred to be low for longer circulating and effective immune modulation. It has been demonstrated that nanoparticles with antifouling zwitterionic group on the surface have long circulation time in human blood with the reduced nonspecific uptake by macrophages. According to an embodiment, 25% of zwitterionic monomer MPC is incorporated into the shell. Immune cell uptake was evaluated in cell culture of both murine macrophage RAW 246.7 and human monocyte THP-1 cells to compare the stealth properties of the core and core-shell nanogels. Nanogels were preincubated with Cy3-α-LA at mass ratio 50:1 for 30 min for complete adsorption as shown in agarose gel electrophoresis (FIG. S10). Then added into the cell culture medium (final concentration of α-LA is 5 μg/mL). The cells were fixed and nucleus were stained with DAPI after 30 min incubation for microscope observation. As show in FIG. 4, the core nanogels with Spm2C17₂ or Arg₄C17₂ nanotraps can be uptaken effectively by both immune cells, due to the positive charges as shown in zetapotential analysis in FIG. 1. The core-shell nanogels significantly reduced the cell uptake, especially for CS(Arg₄C17₂), i.e., core-shell(Arg₄C17₂), with negligible cell uptake, which was confirmed by the quantitative fluorescent intensity analysis on cell signals (FIG. S11). It was also observed that cell morphology changes significantly after nanogel incubation, except for CS(Arg₄C17₂), which is consistent with the cell viability assays (FIG. 3b). Thus, we focused on the core-shell nanogel CS(Arg₄C17₂) in comparison with the core nanogel in the following in vitro and in vivo studies.

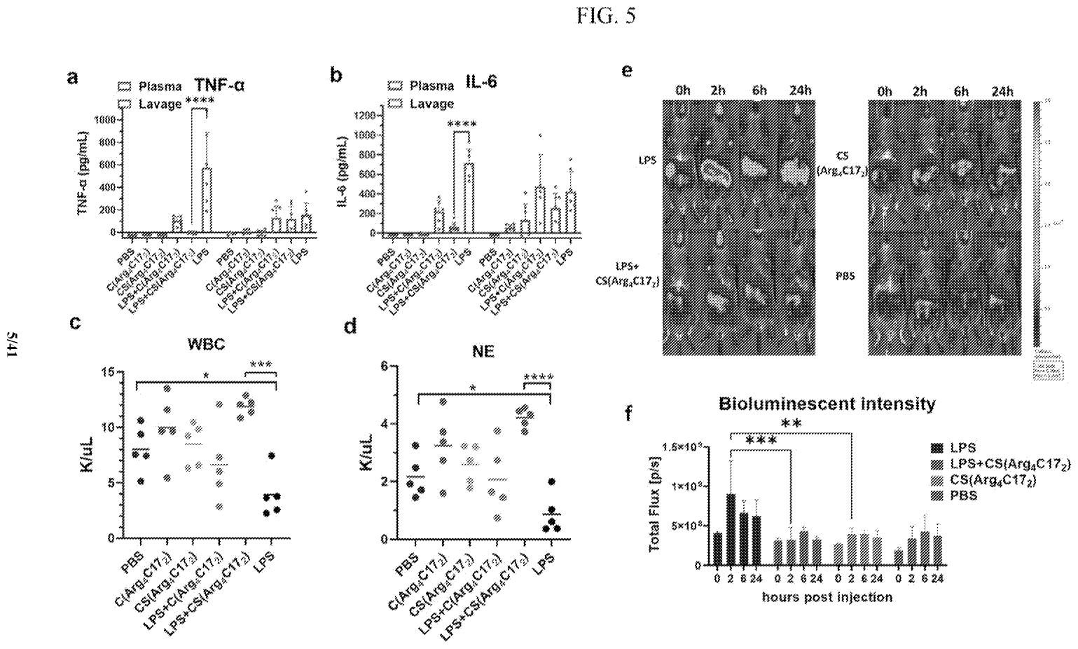

LPS attenuation: LPS, shedding from gram-negative bacteria, can effectively stimulate immune cells to secrete inflammatory mediators, such as tumor necrosis factor-α (TNF-α) and interleukin 6 (IL-6). These proinflammatory cytokines are very potent in mediating inflammatory reactions in control infections and can also cause tissue damage when dysregulated in endotoxemia, thus, can be used as biomarkers for prognosis of sepsis. LPS was co-incubated with core or core cell nanogels contacting nanotraps of Spm2C17₂ or Arg₄C17₂ before added into culture medium of macrophage-like RAW 246.7 cells incubation in comparison with free LPS stimulation. PMB is a potent LPS-binding antibiotics and was used as an agonist for LPS in immune cell stimulation. In addition, PMB was also introduced in the core of the nanogel to compare with our nanotrap nanogels. After overnight incubation, cell culture medium was collected, and the supernatant was obtained for ELISA analysis for TNF-α and IL-6 production (FIG. 5a). As expected, free LPS induced abundant cytokine productions for both TNF-α and IL-6, which can be effectively attenuated by free PMB. The blank nanogels are not immune genetic and did not induce cytokine productions (FIG. 5a). Nanogels functionalized with PMB also exhibited dose-dependent inhibition of TNF-α and IL-6 production significantly compared with free LPS group. Embodiments disclosed herein provide nanotrap-functionalized nanogels with both core and core-shell structures that exhibit much better inhibitory effects for cytokine productions, which is correlated with the stronger LPS binding in nanotrap than PMB. The functionalized core nanogel had a reduced efficacy with the increased nanogel/LPS mass ratio from 100:1 to 200:1 for both nanotrap structures of Spm2C17₂ and Arg₄C17₂, indicating the surface binding of LPS on the core nanogel, which may still present to immune cells for immune stimulation. In contrast, the core-shell nanogels with inert surface exhibited dose-dependent efficacy for LPS attenuation.

LPS binds to TLR-4 receptor triggering intracellular signaling pathways, mainly through nuclear factor-κB (NF-κB) activation to promote inflammatory gel expression. Thus, we apply HIV-LTR/Luciferase (HLL) mice as luciferase reporter for NF-κB activation and inflammatory responses to LPS stimulation with/without nanogels. HLL mice were imaged at t=0 for baseline and at 2, 6, 24 h after i.p. injection of PBS, CS(Arg₄C17₂), LPS or LPS/CS(Arg₄C17₂), respectively (FIGS. 5b and 5c). As shown in the bioluminescent imaging in FIG. 4b, LPS-treated mice exhibited significantly increased luminescent signal in the peritoneal space at 2 h post injection and decreased to almost baseline after 6 h to 24 h, which is correlated with the peak of LPS-induced. However, mice treated with nanogel-treated LPS solution remained the signal low throughout the observation, indication the attenuated activity of LPS. Blank nanogel injection did not induce any inflammatory signals as PBS injection (FIG. 5c).

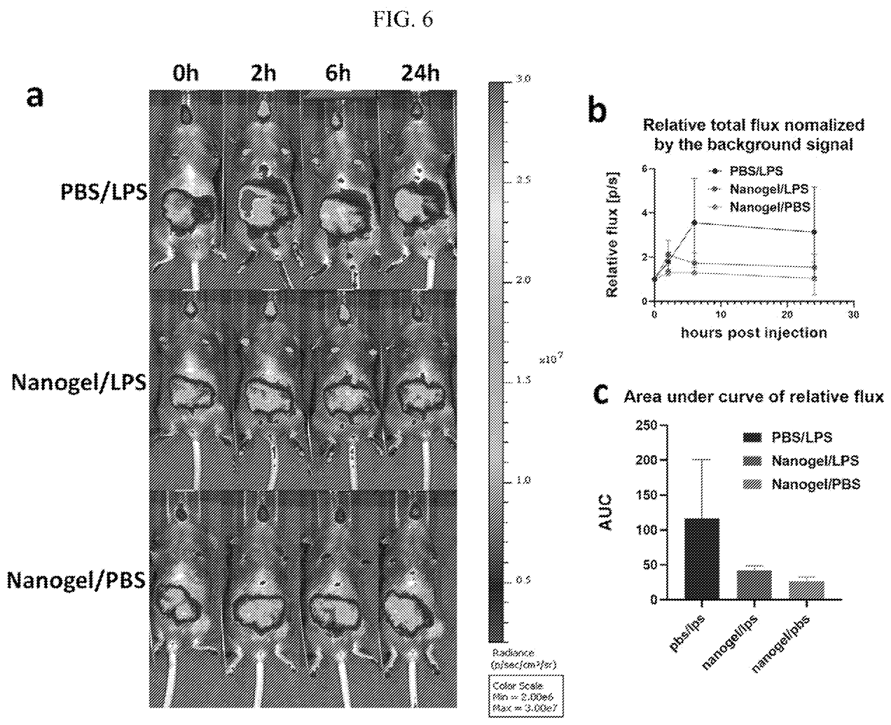

Further, wild type BALB/c mice were applied to evaluate cytokine production after LPS i.p. injection. LPS was pre-incubated with both core and core-shell nanogels, respectively, at mass ratio of 1/100 and then i.p. injected into the mice at a LPS dose of 0.1 mg/kg. The mice were sacrificed 2 h post-injection, plasma and intraperitoneal lavage were collected for cytokine analysis. FIG. 6 shows the levels of the pro-inflammatory cytokines, TNF-α and IL-6, in plasma and lavage. A significant increase in the pro-inflammatory cytokines was observed in free LPS group compared to the control PBS group. Blank core and core-shell nanogels didn't induce significant cytokine production. The LPS/nanogels groups exhibited a significant reduction in cytokine production for both TNF-α and IL-6, especially in plasma, compared to the LPS group. It was consistent with the ex vivo cell culture study, the functionalized core nanogel has less efficacy in attenuating LPS in cytokine stimulation in comparison to the core-shell nanogel. Free LPS did not induced higher cytokine productions in the intraperitoneal lavage than in the blood, which may be due to the quick diffusion of LPS into systemic circulation stimulating white blood cells for cytokine production. It showed that the empty nanogels are not immunogenic. Surprisingly, no significant difference in cytokine productions were observed in the peritoneal groups for animals treated with free LPS or LPS pretreated with both core and core-shell nanogels. However, the significant reduced cytokine production in blood by core-shell nanogel indicate effective LPS attenuation. At the same time, we analyzed the blood counts in mice after sacrificed in FIGS. 6c and 6d, showing significantly reduced both WBC and neutrophile counts in free LPS group, which were effectively attenuated by core-shell nanotrap, whereas, not significant attenuation was observed in mice treated with the nanotrap core structure. As a conclusion, the introduction of an inert shell can effectively shelter the septic molecules adsorbed in the core of nanogel to effectively attenuate the stimulation of inflammation in vivo.

In order to mimic the clinical disease treatment, we apply LPS and nanogel separately through i.p. injection to test the in situ LPS attenuation for inflammation control. We first i.p. injected CS(Arg₄C17₂) nanogel (20 mg/kg) into HLL NF-κB reporter mice with gentle massage to let nanogel homogeneously distributed in the peritoneal cavity. Then, LPS (Pseudomonas aeruginosa, 0.1 mg/kg) was i.p. injected 5 min later. At the same time, PBS was used to replace either LPS or nanogel to generate either negative or positive control for inflammation inductions. As shown in FIG. 6e, the separately administrated core-shell nanogel effectively attenuated the LPS-induced NF-κB activation as reported as bioluminescent signals observed in the abdominal area in LPS treatment mice. The quantitative bioluminescent analysis also revealed the same trend and efficacy for inflammation control (FIG. S12) It confirms that the nanogel had protective effects against LPS-induced inflammation.

Cytokines removal from biological fluids: The management of hyperinflammation is crucial for the treatment of vital sepsis. Inflammatory mediators, such as TNF-α and IL-6, are produced by immune cells in response of infections, which stimulate strong immune reactions and even hyperinflammation if released into the circulation. Most of the proinflammatory cytokines have small molecular weight (10-30 kDa) and negatively charge. In this regard, the nanogels may be able to absorb these cytokines, which has the similar molecular weight and negative charges with LPS. Thus, we collected several cytokine containing solutions for incubation with nanogels to test their efficacy for cytokine adsorption. First, pure TNF-α and IL-6 solutions was incubated with the of core-shell nanogel with (Arg₄C17₂) nanotrap and was effectively removed in a dose-dependent manner with >90% efficiency at a mass ratio of 200:1 evaluated by ELISA (FIG. S13).

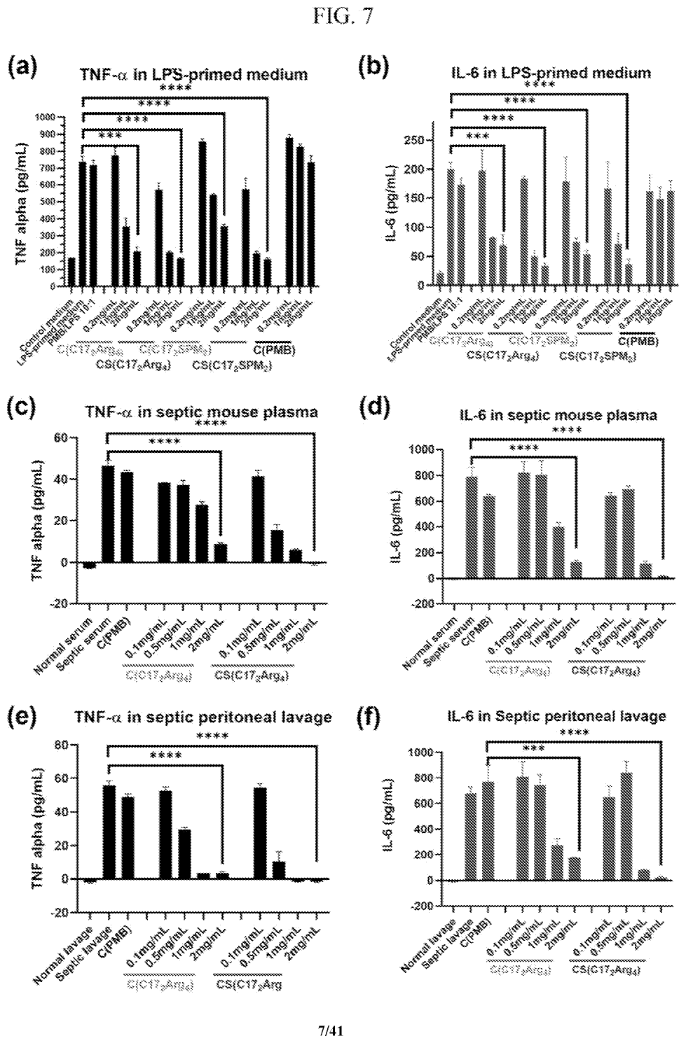

Cell culture medium of RAW264.7 cells after LPS stimulation (50 ng/mL) for 24 h was harvested and incubated with nanogels to characterize efficiency for cytokine adsorption. Significant cytokines, e.g., TNF-α and IL-6, were produced in the cell culture medium after LPS stimulation (FIGS. 7a and 7b). Both core and core-shell nanogels with positively charged (Arg₄C17₂) or (Spm2C172) nanotraps can effectively adsorb both TNF-α and IL-6 in a dose dependent manner as measured by ELISA assays. The core-shell nanogels exhibited better efficacy than the core nanogels. In contrast, nanogel with PMB or free PMB did not show any capacity for the adsorption of these two cytokines (FIGS. 7a and 7b). Further, the plasma and peritoneal lavage fluid collected from sepsis mice 24 h after the cecum ligation and puncture (CLP) surgery for incubation with different concentrations of core and core-shell nanogels, respectively. After overnight incubation, TNF-α and IL-6 level in the fluids were analyzed by ELISA. Similarly, both core and core-shell nanogels can scavenge TNF-α and IL-6 production in a dose-dependent manner, which was significantly better than the PMB-containing nanogel (FIGS. 7c-7f). The adsorption of cytokines in both cell culture media and septic blood and body fluids were more effective after incubation with core-shell nanogels than the core nanogels, which are expected due to the size excusive effects of shell layer preventing the competition of large abundant serum proteins.

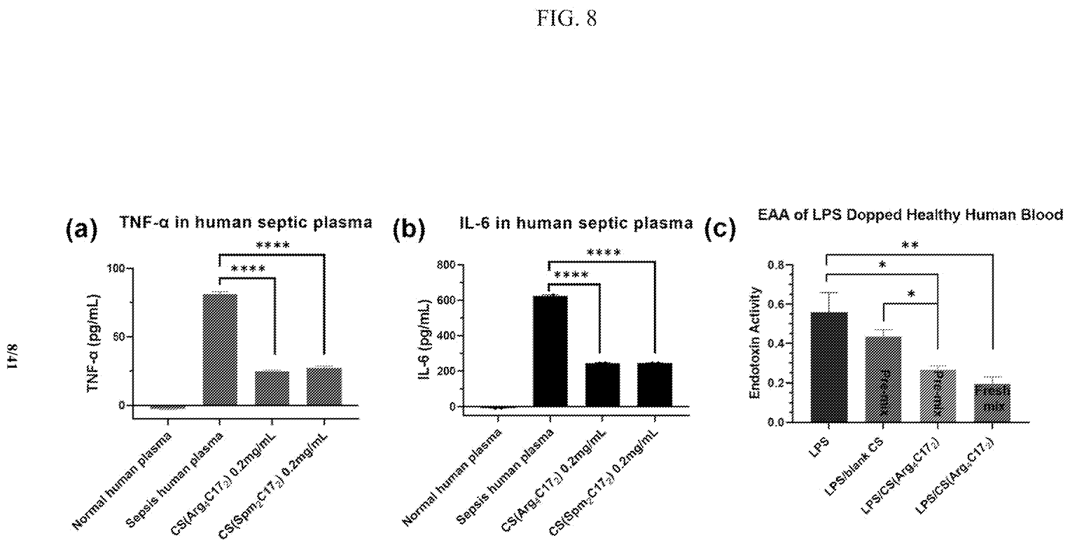

Attenuation of septic molecules in sepsis patient blood: To determine the potential of the nanogel for the treatment of human sepsis patient. The plasma collected from eight de-identified sepsis patients were pooled together and incubated with nanogels for overnight. The cytokine concentrations were analyzed via ELISA assays for plasma with and without nanogel treatment. FIG. 8 showed significantly decrease the levels for both TNF-α and IL-6 after incubation with CS(Arg₄C17₂). LPS and CS(Arg₄C17₂) (mass ratio 1:200) with or without preincubation were added into the healthy patient blood and the endotoxin activity (EA) were evaluated using EAA kit. The addition of nanogel nanotrap can reduce the level of endotoxin. As a comparison the blank nanogel which did not have LPS binding capability cannot reduce the level of endotoxin. stimulatory concentration of endotoxin (E. coli O55:B52.3 ng/ml). Endotoxin activity (EA) was calculated by the following equation: EA=(Tube 2 RLU integral−Tube 1 RLU integral)/(Tube 3 RLU integral−Tube 1 RLU integral). RLU: relative light units.

### Example I

Materials of the Example: N-lsopropylacrylamide (NIPAm, TCI, +99%) was recrystallized by hexane and dried in vacuum before use. Acrylic acid (AAc, +99%), heptadecanoic acid (C17), 2-Methacryloyloxyethyl phosphorylcholine (MPC, +96%) were purchased from TCI. N, N′-methylenebis(acrylamide) (BIS, +99%), Di-tert-butyl decarbonate (Boc20, +98%) and sodium dodecyl sulfate (SDS, +98%) were obtained from Alfa Aesar. Potassium persulfate (KPS) and N-hydroxysuccinimide (NHS, +98%) were purchased from ACROS. Spermine (SPM), Rink amide-MBHA resin (HCRAm 04-1-1) was ordered from Nankai HECHENG S&T Co., Ltd (Tianjin, China). (Fmoc)-Lys(Boc)-OH, (Fmoc)-Lys(Fmoc)-OH, (Fmoc)-Lys(Dde)-OH, trifluoroacetic acid (TFA), N-(3-Dimethylaminopropyl)-N′-ethylcarbodiimide hydrochloride (EDC HCl) were obtained from Chem-Impex International, Inc. (Wood Dale, IL). (Fmoc)-Arg(Pbf)-OH was purchased from AnaSpec Inc. (San Jose, CA). N, N′-diisopropylcarbodiimide (DIC), N-hydroxybenzotriazole (HOBt), succinic anhydride, 4-dimethylaminopyridine (DMAP), N,N-dimethylformamide, anhydrous (DMF, 99.8%) chloroform (CHCl3), methylene chloride (DCM), methanol (MeOH), dimethyl sulfoxide (DMSO) were received from Acros Organics (Belglum, NJ). tert-Butyl bromoacetate, N-hydroxysuccinimide (NHS), triethylamine (TEA), polymyxin B (PMB), hydrazine hydrate, triisopropylsilane (TIS), α-lactalbumin (α-LA from bovine milk), and LPS from Escherichia coli (L4130) were purchased from Sigma-Aldrich (St. Louis, MO). Tetrazolium compound [3-(4,5-dimethylthiazol-2-yl)-5-(3-carboxymethoxyphenyl)-2-(4-sulfophenyl)-2H-tetrazolium, MTS] and phenazine methosulfate were purchased from Promega (Madison, WI). ELISA kits were purchased from companies for direct use (e.g., IL-6 Cat. #: BMS603-2 and TNF-α: Cat. #: BMS607HS from Invitrogen).

Synthesis of nanotraps: Oligomers bearing both positive charge groups and hydrophobic groups were formulated by SPPS following a published procedure. Starting from Rink amide resin (0.5 mmol/g), Fmoc-Lys(Fmoc)-OH, Fmoc-Lys(Dde)-OH, and Fmoc-Arg(Pbf)-OH were coupled sequentially following the standard peptide synthesis procedures. DIC and HOBt were used as catalytic coupling reagents. All the coupling reactants were in three-fold excess to amine functional group on the resin. Fmoc protecting group was removed by the treatment of resin with 20% 4-methylpiperidine solution in DMF for 30 min. De-Dde was carried out in 2% hydrazine DMF solution for 5 min and three times. The completion of the reaction was monitored by the ninhydrin test and confirmed by MALDI-TOF MS of the cleaved compound. After completion, residual reactants were removed by filtration under vacuum and washed with copious solvents of DMF, DCM, and MeOH. The cleavage of dendrons from rink resin was conducted in TFA/TIS/H₂O (95/2.5/2.5, v/v/v) cocktail. The final oligomers were purified by precipitation with cold ethyl ether followed by dialysis against water.

Synthesis of core nanogel: Poly(NIPAm-co-AAc-co-MPC) nanogel was prepared by free radical precipitation polymerization with N,N′-methylene bisacrylamide (BIS) as crosslinker. N-isopropylacrylamide (NIPAm, 900 mg, 7.95 mmol), acrylic acid (AAc, 0.64 mg, 0.0088 mmol), 2-Methacryloyloxyethyl phosphorylcholine (MPC, 260 g, 0.88 mmol), N, N′-methylene bisacrylamide (BIS, 27.2 mg, 0.18 mmol) and sodium dodecyl sulfate (SDS, 30 mg) were dissolved in 100 mL of DI water in a two-neck round-bottom flask. The solution was purged with nitrogen for 1 h at room temperature, at medium stirring rate, and then heated to 70° C. Potassium persulfate (KPS, 23.5 mg, 0.088 mol) was dissolved in 1 mL of DI water and purged with nitrogen for 10 min and then added to the solution to initiate the polymerization. The reaction was maintained at 70° C. under nitrogen for 30 min. The reaction mixture was then cooled, transferred into a pre-washed dialysis tube with molecular cutoff ˜3.5 k and dialyzed for 2 days.

Synthesis of core-shell nanogel: The core-shell nanogel were synthesized via a two-stage seeded polymerization method with the core particle as seeds. 50 mg of poly(NIPAm-co-AAc-co-MPC) was dissolved in 10 mL DI water and sonicated to ensure complete solubilization. The particle suspension was heated at 70° C. and purged with nitrogen for 1 h. NIPAm (20 mg, 0.18 mmol), MPC (13 mg, 0.044 mmol), BIS (1.7 mg, 0.011 mmol), and KPS (0.6 mg, 0.0022 mmol) were dissolved in 2 mL of water and purged with nitrogen for 10 min. 0.3 mL of this solution was added to the core nanoparticle suspension, and the remaining 1.7 mL of solution was added in aliquots of 0.3 mL every 5 min. The reaction was allowed to proceed at a temperature of 70° C. for 3 h. Poly(NIPAm-co-AAc-co-MPC) core-poly(NIPAm-co-MPC) shell particles were dialysis with molecular cutoff ˜3.5 k for 2 days.

Functionalization of core and core-shell nanoparticles: The nanotraps containing amine group were conjugated by condensation to the carboxylic group of AAc present in the poly(NIPAm-co-AAc-co-MPC) core nanogel. A preliminary activation of the carboxylic group present in the nanoparticles was performed. Briefly, 10 mL core or core-shell nanogel suspensions (5 mg/mL) were added 0.84 mg (0.0044 mmol) of EDC HCl and 0.51 mg (0.0044 mmol) of NHS. The reaction was held at room temperature and stirred for 2 h. Then, an appropriate amount of nanotraps (molar ratio of protein binding moieties/acrylic acid 10:1) was dissolved in 1 mL of water and added to the reaction mixture with triethylamine; the reaction was held at room temperature at medium stirring rate for 24 h. In order to remove the unreacted small molecules, the core-shell nanoparticles were dialysis with molecular cutoff ˜3.5 k for 3 days.

Electrophoresis assays: The loading capacities of the nanogel to LPS, α-LA or BSA were investigated using electrophoresis assay. The electrophoresis was carried out in 1.5% agarose gel (Tris-borate-EDTA (TBE) buffer) at constant current of 35 mA for 30 min. The gel was imaged by a Bio-Rad Universal Hood II Imager (Bio-Rad Laboratories, Inc.) under SYBR Green modes or photographed under UV illumination.

Fluorescent polarization assays: The binding of nanogel to LPS were evaluated and compared with polymyxin B (PMB) by fluorescence polarization (FP) using Multi-Mode Microplate Reader (Synergy™ 2, Biotek, VT) equipped with dichroic mirror (510 nm) and polarizing filter. The measurements were carried out on black flat-bottom 96-well plates (Nunclon™ Surface, Roskilde, Denmark). LPS-FITC with different ratios of nanogel or PMB were incubated in the 96-well plate for 1 h. The FP of LPS-FITC was recorded at excitation and emission filter of 485/20 nm and 528/20 nm, respectively. The experiments were performed in triplicate.

Hemocompatibility Assay: Fresh blood from healthy human volunteers was collected and diluted into 5 mL of 20 mM EDTA PBS. Red blood cells (RBCs) were separated by centrifugation at 3,000 rpm for 10 min and then washed three times with 10 mL of PBS. The RBCs were re-suspended in 20 mL of PBS and 200 μL of RBC solution was added into each well of a 96-well plate. The nanogel solutions were added into the RBCs suspensions to final concentrations of 10, 100, and 500 μg/mL and incubated at 37° C. At determined time (0.5 h, 4 h and 24 h), the mixtures were centrifuged at 3,000 rpm for 3 min, and then the hemoglobin in the supernatant was determined by measuring the UV-Vis absorbance at 540 nm (NanoDrop 2000c spectrophotometer, Thermo Scientific). The RBCs incubated with Triton-100 (2%) and PBS were used as positive and negative controls, respectively. The hemolysis ratio of RBCs was calculated using the following formula: Hemolysis %=(ODₛₐₘₚₗₑ−OD_{PBS})/(ODₜᵣᵢₜₒₙ−OD_{PBS})×100%. All hemolysis experiments were carried out in triplicates. Cell Culture and Cell Viability Assay: Murine macrophage-like RAW 264.7 cells, human fibroblast HFF-1 cells and human monocytic THP-1 cells were purchased from American Type Culture Collection (Manassas, VA). The cells were cultured in Dulbecco's modified Eagle's medium (DMEM) medium containing 10% fetal bovine serum (FBS), penicillin G, and streptomycin at 37° C. in a humidified incubatorwith 5% CO₂. The cytotoxicities of nanogel with or without nanotraps were studied by measuring cell viability via MTS assays. Cells were seeded at a cell density of 4×10³ cells per well in 96-well plates. After overnight incubation, nanogels with different concentrations were added in each well to treat the cells. After 72 h incubation, CellTiter 96 AQᵤₑₒᵤₛ MTS reagent was added to each well according to the manufacturer's instructions. The cell viability was determined by measuring the absorbance at 490 nm using a microplate reader (BioTek Synergy H1) with the untreated cells served as negative controls. Results were obtained as the average cell viability of triplicate experiments calculated by a formula of [(ODₜᵣₑₐₜ−OD_{blank})/(OD_{control}−OD_{blank})×100%].

Cellular uptake of nanogels by macrophages: The cellular uptake of core and core-shell nanogels were determined by fluorescence microscopy. α-LA was used as a model protein, which was chemically labeled with fluorescent dye of Cy-3. Raw 264.7 cells (5×10⁴) and THP-1 cells (5×10⁴) were plated 96-well plates in the cell culture medium and incubated overnight. The Cy3-α-LA was incubated with core or core-shell nanogels at mass ratio 1:50 for 1 h. Then, the Cy3-α-LA loaded nanogels were added into the cell culture medium at a final concentration of Cy3-α-LA 5 μg/mL. After half hour incubation, the cell culture medium was removed. The cells were fixed by 4% paraformaldehyde and the nuclei were stained by 4′,6-diamidino-2-phenylindole (DAPI). Finally, the cells were imaged by microscope.

LPS attenuation in vitro: Raw 264.7 Cells were plated in 96-well plates at a density of 2×10⁴ cells/well. Stock solution of LPS (10 μg/mL) was pretreated with nanogels/PMB at different mass ratio for overnight incubation before being added into macrophage cell culture. The untreated stock LPS solution was directly added to the cell culture to a final LPS concentration of 50 ng/m L as a control for cytokine production comparison. After overnight incubation, cell medium was collected, and the supernatant was obtained by centrifugation. The level of TNF-α and IL-6 production were assessed using the commercial ELISA Kit (eBioscience™).

LPS attenuation in vivo: All animal experiments were performed in compliance with the institutional guidelines and according to the protocol approved by the Committee for the Humane Use of Animals of State University of New York Upstate Medical University. BALB/c mice (8-10 weeks or 11 months, both sexes) purchased from Charles River (USA) were maintained under pathogen-free conditions (22±2° C. and 60% air humidity, 12 h light/dark cycle) according to the AAALAC (Association for Assessment and Accreditation of Laboratory Animal Care) guidelines and were allowed to acclimatize for at least 4 days before any experiments.

BALB/c mice were randomly divided into six groups (n=5-7). Group 1: Sham administered with a single intraperitoneal (i.p.) dose of PBS; Group 2,3: Sham associated with core or core-shell nanogel administered i.p. at the dose of 10 mg/kg; Group 4: LPS i.p. administered at the dose 0.1 mg/kg; Group 5, 6: LPS associated with nanogels (pre-incubated for overnight), with a single i.p. administration. Mice were sacrificed after 2 h. The blood was extracted, the peritoneal cavity was rinsed with 600 μL of PBS to collect peritoneal lavage. The cytokine level in plasma and peritoneal lavage were evaluated by ELISA.

8-12 week-old, NF-κB reporter HLL (HIV-long terminal repeat/luciferase) mice were used for in vivo bioluminescence studies. 1 day before the procedure, the abdominal area of the mice were shaved for optimal imaging acquisition. To obtain a basal bioluminescence level of each experimental mice, luciferin solution (200 μL/20 g mice, dose of 150 mg/kg) was i.p. injected, 10 minute later the abdominal bioluminescence was acquired using in vivo imaging system (IVIS) while the mice were under anesthesia through isoflurane inhalation. At the procedure day, LPS (Pseudomonas aeruginosa, dose of 0.1 mg/kg) pre-incubated with core-shell nanogel (dose of 20 mg/kg) or PBS was i.p. injected into mice. Controls were injected with PBS or nanogel in PBS solution. The abdominal bioluminescence signals were acquired using IVIS 2 hours, 6 hours, and 24 hours post injection.

Cytokines removal from biological fluids: Raw 264.7 Cells were plated in 96-well plates at a density of 2×10⁴ cells/well. Stock solution of LPS was directly added to the cell culture to a final LPS concentration of 50 ng/mL. after 24 h coincubation, the cell culture medium was harvested. Septic mice were induced by the cecum ligation and puncture (CLP) procedure as previously described. 24 h after CLP, the sepsis mice plasma and peritoneal lavage were collected for cytokines removal study. To determine nanogels binding with cytokines, including IL-6 and TNF-α, 100 μL of nanogel samples mixed with IL-6 or TNF-α were incubated at 37° C. for overnight. Following the incubation, cytokine concentrations in the supernatant were quantified by using ELISA. The cytokine removal capability of were tested by adding core or core-shell nanogel with different concentration into LPS-challenged cell culture medium, sepsis mice plasma and peritoneal lavage, and sepsis human patient plasma, respectively. After overnight incubation at 37° C., cytokine levels from plasma and peritoneal lavage were measured by ELISA. All experiments were performed in triplicate.

Nanogel prevents LPS induced inflammation in vivo: NF-κB reporter HLL mice were used for in vivo bioluminescence studies. The abdominal area of the mice were shaved 1 day before. At the procedure day, 200 μL (20 mg/kg) core-shell nanogel in PBS solution was i.p. injected into mice, followed by a gentle rub around the abdominal area. PBS with same volume was injected as controls. 5 minutes later, 50 μL LPS (Pseudomonas aeruginosa, dose of 0.1 mg/kg) in PBS solution was i.p. injected into the mice. The abdominal bioluminescence signals were acquired using IVIS 2 hours, 6 hours, and 24 hours post injection.

LPS attenuation of nanogel by endotoxin activity assay (EAA): LPS (5 ng/mL) with or without the overnight preincubation with CS(Arg₄C17₂) (1 μg/mL) were added into 1 mL of healthy human blood. LPS preincubated with PBS or core-shell without nanotrap (CS(COOH), 1 μg/mL) were added into the blood as positive controls. The endotoxin activity in each sample were evaluated using the EAA kit (Spectral Medical INC.).

### Example II

Embodiments discloses herein include a functionalized nanogel composition that is made up of a plurality of nanoparticles and each nanoparticle is comprised of a nano-sized crosslinked hydrogel system. The nanoparticle has spherical morphology and a spherical surface, and within the system there is a plurality of dendritic shaped functionalized telodendrimers that contain a plurality of hydrophobic groups with hydrophilic charged binding moieties attached to the hydrophobic groups. The telodendrimers are attached to a linear polyethylene glycol polymer (PEG) chain by a crosslinking compound having at least two double bonds and located between the linear polyethylene glycol polymer (PEG) chain and the functionalized telodendrimers. The linear polyethylene glycol polymer (PEG) chain extends outward from the surface of the nanoparticle and provides a biomolecule size-excluding nanogel network.

The functionalized nano-sized hydrogel composition, i.e., nanogel (NG), can be prepared via a one-pot precipitation polymerization using biocompatible, biodegradable monomers/crosslinkers and versatile polymerizable hybrid telodendrimer (TD) nanotraps (NTs) for effective septic molecules scavenging. The functionalized nanogel composition with TD NG (i.e., PEGylated TD NG) has spherical morphology with homogenous size distribution around 300 nm. TD NG is stable in plasma and can be degraded in the presence of reducing agents, e.g., TCEP and glutathione. The presently claimed size-exclusive nanogel network and the polyethylene glycol (PEG) surface coating exclude the abundant serum proteins for selective and effective capture of septic molecules, e.g., LPS, TNF-α, IL-1β3, IL-6, etc., in cell culture medium, septic mouse plasma and peritoneal lavage and septic patient plasma. PEGylated TD NG is nontoxic in cell culture, low immune cell uptake, biocompatible after i.v. injection with prolonged circulation time and can be excreted into feces via liver-bile ducts. Systemic administration of PEGylated TD NG significantly inhibits the LPS-induced inflammation revealed by the bioluminescent imaging, indicating NF-κB activation in HLL mice. PEGylated TD NG can effectively attenuate inflammatory molecules in situ for effective immune modulation, which ameliorate tissue damage and improve the survival rate in severe CLP sepsis mouse models.

Embodiments of the present disclosure further include an injectable TD NT hydrogel nanoparticle with size 200-300 nm, wherein the disclosed hydrogel utilizes a polyethylene glycol (PEG) on the surface of the particles that can inhibit the unspecific binding. And intraperitoneal injection of claimed nanogel has been demonstrated effectively attenuate NF-κB activation and cytokine production in LPS-induced septic mouse models.

Materials of the Example: Monomethyl-terminated poly(ethylene glycol) monoamine hydrochloride (MeO-PEG-NH₂·HCl, Mw 5 kDa) was purchased from JenKem Technology USA Inc. Acrylic acid (AAc, 99%), 2-methacryloyloxyethyl phosphorylcholine (MPC, 96%) and heptadecanoic acid (C17, 98%) were purchased from TCI. N, N′-methylenebis(acrylamide) (BIS, 99%), lithium hydroxide (anhydrous, 98%) and succinic anhydride (99%) was obtained from Alfa Aesar. (Fmoc)-Lys(Fmoc)-OH, (Fmoc)-Lys(Dde)-OH, (Fmoc)-Lys(Boc)-OH, trifluoroacetic acid (TFA) were obtained from Chem-Impex International, Inc. (Wood Dale, IL). (Fmoc)-Arg(Pbf)-OH was purchased from AnaSpec Inc. (San Jose, CA). N, N′-diisopropylcarbodiimide (DIC), N-hydroxybenzotriazole (HOBt), N-dimethylformamide, anhydrous (DMF, 99.8%), methylene chloride (DCM), methanol (MeOH), potassium persulfate (KPS) and glutathione (GSH, 98%) were received from Acros Organics (Belglum, NJ). Cystamine dihydrochloride (98%) was purchased from Fluka (Buchs, Switzerland). Triethylene glycol methyl ether methacrylate (TEGMMA, contains MEHQ as inhibitor, 93%), triethylamine (TEA), N,N-Diisopropylethylamine (99%), Tris(2-carboxyethyl)phosphine hydrochloride (TCEP), 2-lodoacetamide, Rhodamine B (95%), hydrazine hydrate, acryloyl chloride, α-lactalbumin (α-LA from bovine milk), and LPS from Escherichia coli (L4130) were purchased from Sigma-Aldrich (St. Louis, MO). Tetrazolium compound [3-(4,5-dimethylthiazol-2-yl)-5-(3-carboxymethoxyphenyl)-2-(4-sulfophenyl)-2H-tetrazolium, MTS] and phenazine methosulfate were purchased from Promega (Madison, WI). ELISA kits were purchased from Invitrogen for direct use.

Synthesis of telodendrimers (TDs): The TDs with charge and hydrophobic groups were synthesized using a solution-phase condensation reaction starting from MeO-PEG^{5k}-NH₂ HCl (5 kDa) according to the previous publications.^{14,15} N-terminal-protected lysine was used to build the branched scaffold of TD with HOBt and DIC as coupling reagents. The peptide coupling reactions were carried out with a 3-fold excess of building block and coupling reagents in DMF. The completion of coupling reactions was determined by the Kaiser test. Fmoc-protecting groups were removed by the treatment with 20% (v/v) 4-methylpiperidine in DMF for 30 min, Dde-protecting groups were deprotected in 2% hydrazine DMF solution for 5 min and three times, Boc- and Pbf-protecting groups were removed in TFA/DCM (50/50, v/v) for 30 min. The double bonds were introduced into the TD through the reaction between acryloyl chloride and primary amine with TEA as acid-binding agent. All polymer products were purified by precipitation in chilled ethyl ether and washed for twice and then dried under vacuum. The resulting TDs were further purified by dialysis (molecular cutoff ˜3.5 kDa) against deionized water for 2 days, and then dried by lyophilization.

Synthesis of disulfide crosslinker N, N′-bis (acryloyl) cystamine (BAC). Cystamine dihydrochloride (1 g, 4.44 mmol) was dissolved in 20 mL distilled water. NaOH (10 M, 2 mL) and acryloyl chloride (1.2 g 13.32 mmol, 10 mL DCM) were added dropwise simultaneously under vigorous stirring at 0° C. After the addition, the solution was then maintained at room temperature for overnight. The resulting precipitate was extracted three times with 100 mL of DCM and washed three times with distilled water (20 mL). Finally, BAC was recrystallized from ethyl acetate and vacuum drying. The ¹H and ¹³C NMR spectra of BAC in DMSO-d₆ were shown in FIG. S1. ¹H NMR (600 MHz, DMSO-d₆) δ 8.37 (t, J=5.7 Hz, 2H), 6.28 (dd, J=17.1, 10.2 Hz, 2H), 6.15 (dd, J=17.1, 2.2 Hz, 2H), 5.66 (dd, J=10.2, 2.2 Hz, 2H), 3.48 (q, J=6.4 Hz, 4H), 2.88 (t, J=6.8 Hz, 4H). ¹³C NMR (151 MHz, DMSO) δ 165.22, 132.06, 125.83, 38.41, 37.56. TLC-MS (ESI, Advion): Exact mass calcfor C16H1904S [M-Na]: 283.05, found 283.2.

Synthesis of nanogel: TEGMMA (100 mg, 0.43 mmol), BAC (4.48 mg, 0.017 mmol, pre-dissolved in 200 μL methanol) and TD were dissolved in 10 mL DI water in a two-neck round-bottom flask. The mixture was purged with nitrogen for 30 min at room temperature at stirring rate 500 rpm and then heated to 70° C. An aqueous solution of KPS (1.16 mg, 0.0043 mmol, 100 μL) was added to initiate the reaction. The reaction was allowed to proceed for 1 h under the protection of nitrogen, stopped by cooling the product to room temperature. The nanogel was purified by ultracentrifuge (50,000 PRM, 30 min, 15° C.) twice to remove the unreacted TD and monomer. The particle pellet was resuspended in 10 mL of DI water, transferred into a pre-washed dialysis tube with molecular cutoff ˜3.5 kDa and dialyzed for 2 days. The same procedures were applied to the preparation of nondegradable nanogel (NG-BIS) but using BIS (2.66 g, 0.017 mmol), instead of BAC, as a crosslinker.

Redox degradation of nanogel: The BAC containing nanogel was incubated in PBS (pH 7.2) containing TCEP (10 mM) and 2-lodoacetamide (10 mM) or GSH (10 mM) at 37° C. for degradation study. The change of hydrodynamic diameter (redox sensitivity) of nanogel was monitored by dynamic light scattering (DLS, Zetasizer Ultra, Malvern) measurement at determine time point. The morphology of the nanogel before and after treatment with reductant at different time point were observed by transmission electron microscope (TEM, JEM-1400) with uranyless staining.

Agarose gel electrophoresis assays: The loading capacities of the nanogel to LPS or α-LA were determined using electrophoresis assay. The nanogel and LPS/α-LA were preincubated in PBS or 50% FBS solution for 2 h at different ratio. Samples with loading buffer (30% glycerol aqueous solutions) were loaded into 1.5% agarose gel in Tris-acetate-EDTA (TAE) buffer. The electrophoresis was carried out at constant current of 45 mA for 30 min. The gel was imaged by a Bio-Rad Universal Hood II Imager (Bio-Rad Laboratories, Inc.).

Hemocompatibility assay: The study was approved by the SUNY Upstate Institutional Review Board (IRB #754811). 1 mL of fresh blood obtained from healthy human volunteers was added into 5 mL of 20 mM EDTA PBS. The red blood cells (RBCs) were collected by centrifugation at 3,000 rpm for 10 min and washed three times with 10 mL of PBS and suspended in 20 mL of PBS. 120 μL of RBC solution was mixed with nanogel solutions at final concentrations of 10, 100, 500 and 1000 μg/mL by gentle pipette and incubated at 37° C. for 0.5 h, 4 h, and 24 h. Triton-100 (2%) and PBS were added into RBCs as positive and negative controls, respectively. The mixtures were centrifuged (3,000 rpm, 3 min) and hemoglobin in the supernatant was determined by measuring the UV-Vis absorbance at 540 nm (NanoDrop 2000c spectrophotometer, Thermo Scientific). The percent hemolysis of RBCs was calculated using the following formula: Hemolysis %=(ODₛₐₘₚₗₑ−OD_{PBS})/(ODₜᵣᵢₜₒₙ−OD_{PBS})×100%.

Cell culture and MTS assay: Murine macrophage-like RAW 264.7 cells, human monocytic THP-1 cells and human fibroblast HFF-1 cells were purchased from American Type Culture Collection (Manassas, VA). RAW 264.7 and HFF-1 cells were cultured in DMEM medium, THP-1 cells were cultured in RPMI 1640 medium, supplemented with 10% fetal bovine serum (FBS), 100 U/mL penicillin G and 100 mg/mL streptomycin at 37° C. in a humidified incubator with 5% CO₂. Cells were seeded into 96-well plates at a cell density of 3×10³ (RAW 264.7, HFF-1) and 8×10³ (THP-1) cells per well. After overnight incubation, nanogels with different dilutions were added in each well. After 72 h incubation, the mixture solution composed of CellTiter 96 AQᵤₑₒᵤₛ MTS reagent was added to each well. The cell viability was determined by measuring the absorbance at 490 nm using a microplate reader (BioTek Synergy H1) with the untreated cells served as negative controls. Results were shown as the average cell viability [(ODₜᵣₑₐₜ−OD_{blank})/(OD_{control}−OD_{blank})×100%] of triplicate wells.

Cellular uptake of nanogels by macrophages: Raw 264.7 (1×10⁴ cells/well) were seeded into 96-well plates in DMEM medium and cultured overnight. The FITC-α-LA were incubated with TD (mass ratio 1:5), nanogel (mass ratio 1:50) and PEI (mass ratio 1:2) for half hour and added into cell culture medium at a final concentration of FITC-α-LA 5 μg/mL. After 2 h incubation, the cell culture medium was removed, the cells were washed three times by cold PBS and fixed by 4% paraformaldehyde for 10 min. The cell nuclei were stained by 4′,6-diamidino-2-phenylindole (DAPI). Finally, the cells were imaged by fluorescence microscope.

LPS attenuation in vitro: Raw 264.7 Cells were plated in 96-well plates at a density of 2×10⁴ cells/well. Stock solution of LPS (50 μg/mL) was pretreated with nanogels/PMB/TD at different mass ratio for overnight or added separately (nanogel, then LPS) into cell culture medium. The untreated stock LPS solution was directly added to the cell culture to a final LPS concentration of 50 ng/mL as a control for cytokine production comparison. After overnight incubation, cell medium was collected, and the supernatant was obtained by centrifugation. The level of TNF-α and IL-6 production were assessed using the commercial ELISA Kit.

Experimental animals: Wild type mice (C57BL/6, 6-8 weeks) purchased from Charles River (USA) were maintained under pathogen-free conditions (22±2° C. and 60% air humidity, 12 h light/dark cycle) according to the AAALAC (Association for Assessment and Accreditation of Laboratory Animal Care) guidelines and were allowed to acclimatize for at least 4 days before any experiments. The NF-κB reporter HLL (HIV-long terminal repeat/luciferase) mice colony were originally provided by Dr. Timothy Blackwell-Vanderbilt University Medical Center (also available in the Jackson laboratory #027547) and maintained by our laboratory staff. Briefly, we maintained the colony by crossing hemizygous mice to their wildtype siblings, or to C57BL/6J. The offspring had both hemizygous mice and wildtype mice with a theoretical ratio of 1:1. Their genotypes were confirmed by the presence of NF-κB-induced bioluminescence by injection of luciferin (200 μL/20 g mice, 150 mg/kg) when the mice were 3-4 weeks old at the steady state. 8-12-week-old HLL mice were used for in vivo bioluminescence studies. All animal experiments were performed in compliance with the institutional guidelines and according to the protocol approved by the Committee for the Humane Use of Animals of State University of New York Upstate Medical University (IACUC #437).

Pharmacokinetic PK and biodistribution study: The blood circulation of nanogel was studied using Rhodamine B (RB) labeled nanogel on wild type mice (n=3 per group). The nanogel were synthesized by TD containing RB (PEG^{5k}RB₂). The nanogel was intravenously injected into mice at a dose of 50 mg/kg. As comparison, the PEG^{5k}RB₂ was i.v. injected at with equivalent amount of RB in nanogel (the concentration were quantified by fluorescent signal). At pre-determined time after injection, blood samples were collected from the tail vein, and plasma was separated by centrifugation (7000 rpm, 5 min) for quantitative measurement. The fluorescent signal of RB was recorded at excitation/emission of 525/580 nm on a fluorescence microplate reader Synergy H1 (BioTek Instruments Inc., Winooski, VT). After 28 h, the mice were sacrificed. The organs of the mice were harvested and homogenized with 2× of PBS, followed by centrifugation at 10,000 rpm for 5 minutes. The fluorescent signal in supernatant were recorded on fluorescence microplate reader.

LPS attenuation in vivo: Wild type mice were randomly assigned in groups (n=3-5). NG treatment after i.v. injection of LPS: mice were i.v. injected with LPS at the concentration of 0.1 mg/kg. At 5 min after the injection, PBS or NG (50 mg/kg) was i.v. injected into the animals. NG treatment before i.v. injection of LPS: mice were i.v. injected with PBS or NG (50 mg/kg). 5 min later, LPS (0.1 mg/kg) was i.v. injected into the animals. At 2 h and 4 h after LPS injection, blood samples were collected from the tail vein, and plasma was separated by centrifugation (7000 rpm, 5 min) for ELISA measurement.

Real time in vivo imagining of mice after LPS injection: The NF-κB reporter HLL mice were used for in vivo bioluminescence studies. To obtain a basal bioluminescence level of each experimental mice, luciferin solution was i.p. injected, 10 minute later the abdominal bioluminescence was acquired using in vivo imaging system (IVIS) while the mice were under anesthesia through isoflurane inhalation. At the procedure day, LPS (dose of 0.1 mg/kg) was i.v. injected into mice. The nanogel (50 mg/kg) was i.v. injected into mice 5 min before or after LPS injection. Controls were injected with PBS, nanogel (50 mg/kg) or LPS (0.1 mg/kg) in PBS solution. The abdominal bioluminescence signals were acquired using IVIS 50 (PerkinElmer) 2 h, 4 h, 6 h, 24 h and 48 h post injection.

Cytokinines removal from biological fluids: Raw 264.7 Cells were plated in 96-well plates at a density of 2×10⁴ cells per well. Stock solution of LPS was directly added to the cell culture to a final LPS concentration of 50 ng/mL. After 24 h coincubation, the cell culture medium was harvested. Septic mice were induced by the cecum ligation and puncture (CLP) procedure as previously described.¹² 24 h after CLP, the sepsis mice plasma and peritoneal lavage were collected for cytokines removal study. Clinical surgical sepsis patient blood was obtained in EDTA tube and de-identified for this study under a protocol approved by SUNY Upstate Institutional Review Board (IRB #1321635-7]). Plasma was isolated for incubation with nanogel. To determine nanogels binding with cytokines, 100 μL of nanogel samples mixed with pure IL-6, TNF-α or IL-1 at different mass ratio and incubated at 37° C. for overnight. Following the incubation, cytokine concentrations in the supernatant were quantified by using ELISA. The cytokine removal capability was tested by adding different concentration of nanogel into LPS-challenged cell culture medium, sepsis mice plasma and peritoneal lavage, and sepsis human patient plasma, respectively. After overnight incubation at 37° C., cytokine levels from cell culture medium, plasma and peritoneal lavage were measured by ELISA. All experiments were performed in triplicate.

Sepsis model induced by CLP in mice: For CLP procedure, mice were anesthetized using intraperitoneal ketamine/xylazine (100 mg/kg ketamine, 10 mg/kg xylazine) injection. After adequate anesthesia, the lower quadrants of the abdomen were shaved, and the surgical area was disinfected. A longitudinal midline incision was made using scissors to extend the incision into the peritoneal cavity. After fascial, intramuscular, and peritoneal incision, the cecum was located and exteriorized. The cecum was tightly ligated with a 1.0 suture (COATS, ART 230 A) at about 1.3 cm to the distal end and was perforated into two holes with a 22-gauge needle. One hole is 0.5 cm to the distal end of the cecum and the other hole is 0.5 cm near the ligation side. The cecum was then gently squeezed to extrude a small amount of feces (about 1 mm³) from the perforation sites. The cecum was returned to the peritoneal cavity, and the peritoneum and skin are closed with 5.0 silk sutures. In sham group, mice were operated following the same protocol without CLP procedure. After operation, mice were resuscitated with 1 mL of warmed saline immediately. The animals were returned immediately to a cage with exposure to an infrared heating lamp for 30 min-1 h, until recovery from anesthesia. Mice were provided with free access to food and water in the bottom of the cage. Buprenorphine (0.05 mg per kg, SQ) was injected for postoperative analgesia every 12 h.

Following the above standard CLP procedure, CLP mice were randomly assigned in two groups (n=8-10) for the treatments with nanogel and saline. Five doses of nanogel (50 mg/mL) or saline were administrated at 1 h, day 2, day 3, day 4 and day 5 after CLP surgery. Animals were monitored 14 days for mortality comparison, and body weight and temperature were monitored over time.

Cytokine analysis and histological examination: Following the same CLP procedure, the mice were treated with nanogel at a dose of 50 mg/mL, the mice treated with saline were used as control group. In addition, a sham group was included with a laparotomy procedure with cecum taken out and put back into the abdomen before wound closure. 24 h post CLP, about 100 μL blood were collected from the tail vein, and plasma was separated by centrifugation (7000 rpm, 5 min) for the cytokines study using ELISA. At the end point of the study (14 days), mice were sacrificed under anesthesia (ketamine: 100 mg per kg, xylazine: 10 mg per kg, IM). Heart, liver, kidney, spleen, and intestine were harvested. The tissue was fixed by 10% neutral buffered formalin or frozen in OCT cryo-embedding medium for histologic study. In order to analyze pulmonary structure-function relations, right-side lung lobe was fixed by formalin infusion into the cannulated main bronchus and was immersed in a container of formalin for at least 24 h. Then, the fixed lung tissue was embedded in paraffin for sectioning (5 μm) and then stained with hematoxylin and eosin (H&E) for histopathology analysis.

Statistical analysis: All data points referred to the mean±standard deviation (SD) and were based on at least three separate experiments (n=3). All statistical tests were performed by GraphPad Prism using one-way analyses of variance (ANOVAs) fortwo-group and multiple-group analyses. Statistical significance was represented as *: p<0.05; **: p<0.01; ***: p<0.001; ****: p<0.0001.

Synthesis and characterization of NG-TD: The lipopolysaccharide (LPS), released from the cell wall of gram-negative bacteria, is predominant pathogenesis involved in primary endodontic infections. The LPS possess two negative charge groups and hydrophobic lipid moieties, which could be neutralized and sequestered with molecules containing positively charge and hydrophobic groups. According to embodiments, we have developed a well-defined linear-dendritic telodendrimer (TD) nanoplatform and optimization for both small-molecular drug and protein deliveries. The TDs are conjugated with positive charges and hydrophobic groups in the nanogel (NG), which provides selective loading to smaller sized LPS and proinflammatory cytokines.

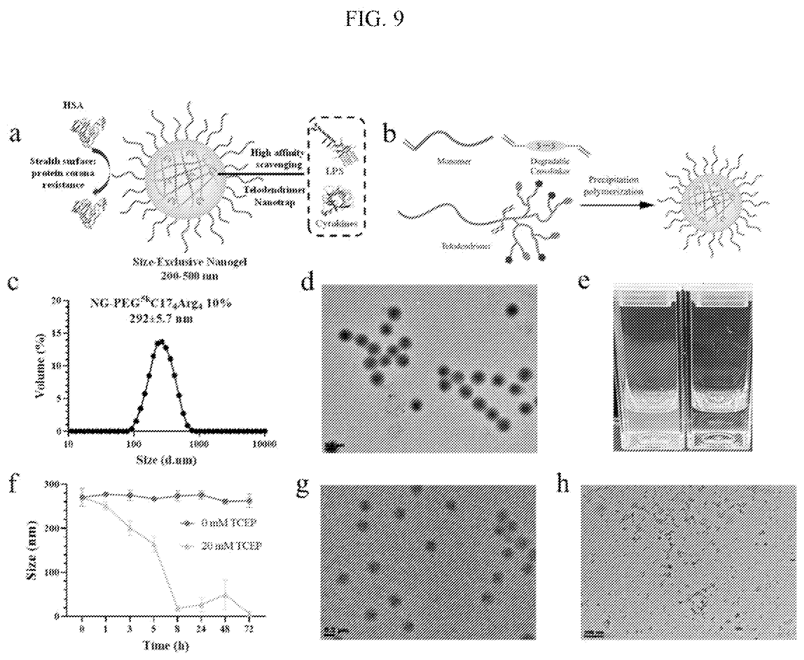

According to embodiments disclosed herein TDs with hydrophobic groups (heptadecanoic acid, C17) and different hydrophilic charge groups (arginine (Arg), oxalic acid (OA) and succinic acid (SA)) have been included in nanogel by radical precipitation polymerization. To incorporate the functional TDs into nanogel, two double bonds were added between the linear PEG chain and dendritic functional group of TD. During the polymerization, TDs can serve as macromer and can be immobilized into the nanogel network by covalent bond (FIG. 9b). We used the rhodamine B labeled TD to test the release profiles of TD from NG and determine the stability of TD in the NG. The RB labeled TD without double bond was made as comparison (FIG. S15). The TD without double bonds showed a fast release from nanogel, around 40% of the TD release out after 48 h, due to the TDs were physically trapped in the crosslinking network. The TD with double bond can decrease the release and increasing the crosslinking degree from 2% to 4% can further reduced the TD release kinetics. Based on this result, we made nanogels using TDs with double bonds and 4% crosslinking degree for the further study. Nanogels were monodisperse, with an average diameter of 200-400 nm in phosphate-buffered saline (PBS) (Table 1 and FIG. 14c and FIG. S16). TEM micrographs showed that the nanogels maintained a uniform spherical morphology and homogeneous size distribution (FIG. 9d). During the polymerization, the hydrophilic linear part of the TDs tends to locate on the surface of the nanogel to stabilize the particle. The TD with arginine (Arg) showed significant charge positive charge (zeta-potential: 30.7±0.6 mV, Table S1) in aqueous solution. With the ratio of PEG^{5k}C17₄Arg₄ less than 10%, the NGs showed neutral surface (Table 1). However, when further increase the ratio of TD into 15% and 20%, the NGs exhibited slightly positive surface, with zeta-potential 3.4±0.3 and 3.5±0.07 mV, respectively. It may be due to as the increase of the number of TDs, there were more positively charged arginine groups approach to the surface of the nanogels. We also made TDs with negative charged oxalic acid (OA) and Succinic acid (COOH) for comparison. The TDs only show negative charges (Table S1), however, the NG-TD at 10% of TD showed neutral surface (Table 1).

TABLE 1
TD composition, size, PDI, zeta-potential and loading capacity of NGs.

| | | Size | | Zeta | Loading capacity |
|---|---|---|---|---|---|
| Entry | Feed ratio of TD (wt %) | (d · nm) | PDI | potential (mV) | of LPS |
| 1 | 0% | 361 ± 3 | 0.03 ± 0.04 | −0.4 ± 0.1 | — |
| 2 | PEG^{5k}-C17₄Arg₄(5%) | 321 ± 10 | 0.02 ± 0.01 | −0.7 ± 0.08 | <1% |
| 3 | PEG^{5k}-C17₄Arg₄(7.5%) | 202 ± 0.8 | 0.4 ± 0.01 | 0.0 ± 0.4 | 1% |
| 4 | PEG^{5k}-C17₄Arg₄(10%) | 226 ± 17 | 0.2 ± 0.04 | −0. ± 0.2 | 4% |
| 5 | PEG^{5k}-C17₄Arg₄(15%) | 266 ± 22 | 0.2 ± 0.007 | 3.4 ± 0.3 | 4% |
| 6 | PEG^{5k}-C17₄Arg₄(20%) | 246 ± 7 | 0.1 ± 0.03 | 3.5 ± 0.07 | 4% |
| 7 | PEG^{5k}-C17₄COOH₄(10%) | 256 ± 5 | 0.1 ± 0.03 | −0.7 ± 0.1 | — |
| 8 | PEG^{5k}-C17₄OA₄(10%) | 348 ± 10 | 0.06 ± 0.05 | −1.5 ± 0.9 | — |

The TD structures can be found, e.g., in FIG. S14-S26. Data represent the means±SD, n=3.

TABLE S1
Zeta-potential of telodendrimers measured by DLS in water.

| | Zeta potential (mV) |
|---|---|
| PEG^{5k}-C17₄Arg₄ | 30.7 ± 0.6 |
| PEG^{5k}-C17₄SA₄ | −20.7 ± 4.3 |
| PEG^{5k}-C17₄OA₄ | −22.9 ± 0.1 |

Biodegradation of nanogel (NG): The incorporation of disulfide crosslinker BAC offers reduction triggered degradability to the nanogel. The response of BAC containing nanogel (NG-BAC) to reducing agents was evaluated by investing their size as a function of GSH and TCEP treatment. The nanogel with undegradable crosslinker BIS (NG-BIS) were incubated with reductant as control. In the presence of 10 mM TCEP, the NG-BIS kept its size and morphology even after 72 h incubation (FIG. S17a-c). Unlike NG-BIS, after 1 h incubation with 10 mM GSH at 37° C., the size of NG-BAC decreased from 285±10 to 245±4 nm (FIG. S17d-f). At 24 h, the size of NG-BAC increased to around 400 nm. The reductant can cleave the disulfide bond into thiol groups, which may further form disulfide bonds through thiol-disulfide exchange reaction and form large aggregations. To demonstrate this hypothesis, we tried to use TCEP and 2-iodoacetamide into the nanogel solutions. The 2-iodoacetamide can react with newly formed thiol group and inhibit the aggregation. The size of the NG-BAC gradually decreased when treated with TCEP and 2-iodoacetamide, 164±18 nm at 24 h and 19±6 nm at 48 h by DLS (FIG. 9f). The size change of NG-BAC was also confirmed by TEM (Hydrodynamic sizes of nanogel with undegradable crosslinker (NG-BIS) with or without incubation with 10 mM TCEP at different time point; the morphology of (b) NG-BIS and (c) NG-BIS incubated with 10 mM TCEP for 72 h by TEM imaging (FIGS. 9g and 9h).

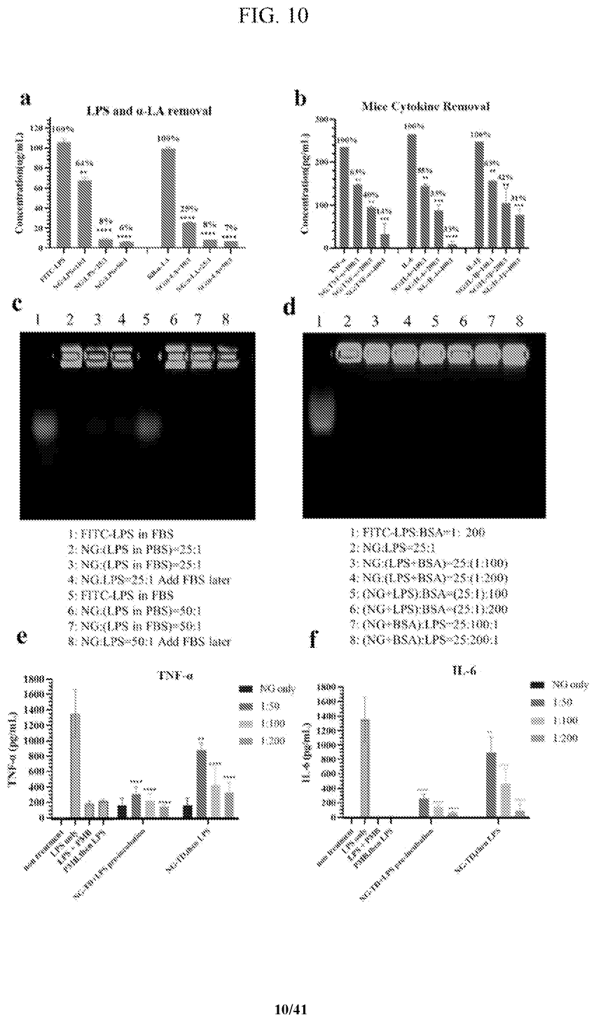

The loading of NG-TD to LPS and proinflammatory cytokines: According to our previous study, the TD can be freely customized by different functional groups for efficient protein encapsulation through synergistic hydrophobic and charge interactions. Here we made the TD with hydrophobic C17 and hydrophilic charged group Arg, OA or SA into the TD for the LPS and proinflammatory cytokines. From the agarose gel result (FIG. S18a), the TD with positively charged Arg groups show efficient loading to negatively charged LPS. In contrast, the OA and COOH containing TDs with negatively charge can't load LPS. Without the incorporation of TDs, the blank nanogel didn't exhibit any effective capture for both proteins (α-LA) and LPS (FIG. S18b). Then we tested the loading capacity of LPS using nanogel with different ratio of PEG^{5k}C17₄Arg₄ by agarose gel electrophoresis. As show in Table 1 and FIG. S19, at 100/1 mass ratio of NG to LPS, the NG-5% TD show partial loading to LPS, however NG-7.5% TD can completely absorb the LPS, with loading capacity 1%. When increasing the TD to 10%, the loading capacity of the LPS can be increased to 4%. However, further increasing the TD to 15% and 20% didn't show further increase of the loading, which due to the TD may aggregate and self-assemble into micelle at high density resulting in the hindering of the molecules to diffuse into the NG. In the contrary, the nanogel with negatively charged TD (OA and COOH) didn't exhibit any scavenge capability to LPS (FIG. S20). We then further confirm the loading capacity of NG-TD (10% PEG^{5k}C17₄Arg₄) to LPS and small-negatively charged model protein α-LA by centrifugation method (FIG. 10a). The NG were preincubated with different ratio of FITC-LPS or RB-α-LA for 2 h. Then the samples were centrifuged (50,000 rpm, 1 h, 15° C.) to separate and determine the concentration of the free LPS or protein in supernatants. As shown in FIG. 2a, more than 90% of LPS and α-LA can be loaded at 25/1 NG to LPS/α-LA ratio, which is consistent with the agarose gel result.

Most proinflammatory cytokines (TNF-α, IL-1, IL-6, IL-12, and HMGB-1) have negative charges with Pls ranging between 4.1 and 6.4. In order to determine the cytokine absorption property of NG, we coincubated the NG-TD (10% PEG^{5k}C17₄Arg₄) with proinflammatory cytokines (TNF-α, IL-6 and IL-1β) at different mass ratio at 37° C. for overnight and then test the free cytokines level by ELISA. As shown in FIG. 10b, at 400:1 mass ratio of nanogel to cytokines, around 86%, 97% and 69% of TNF-α, IL-6 and IL-1p can be removed, which indicate the potential of nanogel for endotoxin and pro-inflammatory cytokines absorption in blood. Base on this result, we chose the Arg containing NG, NG-TD (10% PEG^{5k}Arg₄C17₂) for our further study.

To investigate the ability of the NG to scavenge LPS or cytokines in biofluid, LPS was doped into the fetal bovine serum (FBS, 50%) solution and then incubated with NG. Even through there are plenty of proteins in FBS solution, the nanogel maintains good loading of LPS. From the agarose gel result, the LPS can be completely removed at 25 to 1 mass ratio of NG to LPS (FIG. 10c, lane 3). We also try to first incubate the NG and LPS (lane 4), then added the FBS to challenge the LPS loading. The addition of the FBS didn't influence the loading of LPS. In addition, the negatively charged protein α-LA (mw: 14 kDa) was chosen as the model protein to mimic the proinflammatory cytokines (FIG. S21). The α-LA can be completely loaded at 25:1 mass ratio of NG to protein both in PBS and FBS and the α-LA/NG complex was stable in FBS solution. We also used large amount of bovine serum albumin (BSA) to challenge the loading and determine the selectivity of the NG, Albumin is negative charged high-abundance serum proteins but have larger molecular weight (66 kDa). We tried to add the NG in to the mixture of LPS and BSA or add BSA before or after LPS addition. As shown in FIG. 10d, the addition of excess BSA (100× and 200× to LPS) didn't influence the loading capacity of nanogel to smaller endotoxin.

LPS can activate Toll-like-receptor 4 (TLR4), which induces the production of inflammatory mediators (e.g. TNF-α and IL-6) and triggers cytokine storm. The uncontrolled release of cytokines is the major driving force of sepsis. Here, LPS was co-incubated with NG-TD and then added into culture medium for macrophage-like RAW 264.7 cells. After overnight incubation, cell culture medium was collected for the TNF-α and IL-6 production analysis. The free LPS stimulation were used as positive control. Polymyxin B (PMB), a cationic LPS-neutralizing antibiotic, was used as comparison. As expected, TNF-α and IL-6 productions were significantly increase in free LPS-induced cell, however, it can be effectively attenuated by PMB (FIG. 10e, and FIG. 10f). The blank NG didn't induce cytokine production. NG showed dose-dependent LPS attenuation capability, and can significantly inhibit the cytokines secretion from macrophages. In order to mimic clinical disease treatment, we added LPS and nanogel separately into the cell culture medium to further determine if NG can binding with LPS in situ to inhibit cytokine production for inflammation control. As shown in FIG. 10e, and FIG. 10f, even though the inhibition was not as effective as preincubation groups, at high ratio of NG/LPS (100/1 and 200/1), the NG can also effectively attenuate the LPS-induced proinflammatory cytokines production. This result was as expected, the complex component in cell culture medium may influence the LPS loading capacity of NG. But the NG still maintained high loading capacity to negatively charged smaller protein and LPS, which consistent with the previous agarose gel result.

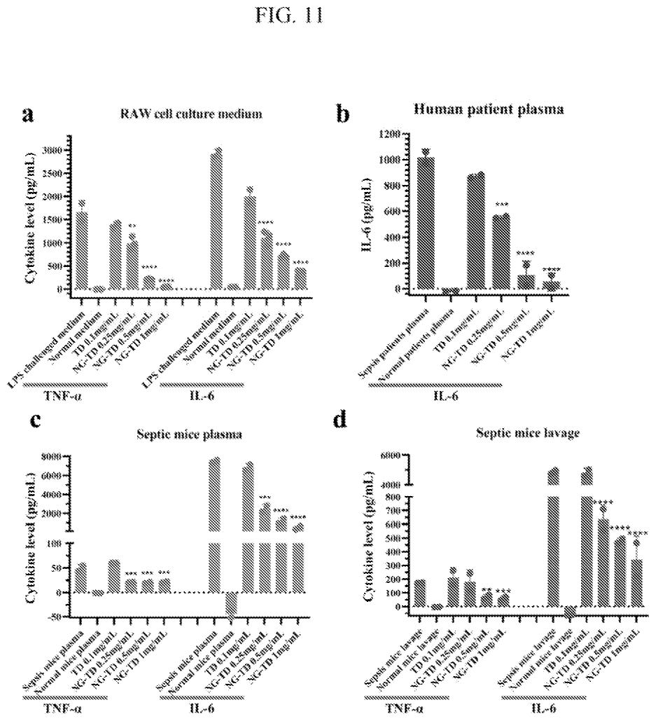

Cytokine Removal from Biological Fluids: The management of hyperinflammation is crucial for the treatment of sepsis. Based on the previous study, we hypothesized that the NG-TD may be able to sequester proinflammatory cytokines and inhibit their ability to potentiate the sepsis cascade. To demonstrate this, we collected cytokine containing biological fluids, including LPS stimulated RAW264.7 cell culture medium, plasma and peritoneal lavage from septic mice and plasma from human sepsis patients, and incubated with NG for cytokine adsorption analysis. As shown in FIG. 11a, LPS stimulation significantly induce the cytokines, e.g., TNF-α and IL-6, production the cell culture medium. The NG with positively charged TD (PEG^{5k}-Arg₄C17₄) nanotraps can effectively adsorb both TNF-α and IL-6 with a dose dependent manner after overnight incubation. In contrast, TD only or nanogel with negatively charge (PEG^{5k}-OA₄C17₄ and PEG^{5k}-COOH₄C17₄) didn't show any capacity for the adsorption cytokines (FIG. S22). We also collected the plasma and peritoneal lavage fluid from septic mice, and incubated with different concentrations NG, TNF-α and IL-6 level in the fluids were analyzed by ELISA. Similarly, nanogels can scavenge those proinflammatory cytokines level in a dose-dependent manner (FIG. 11c and FIG. 11d). Further, the plasma from human sepsis patients were collected for nanogel incubation, there is significant decrease of IL-6 after incubation NG (FIG. 11b).

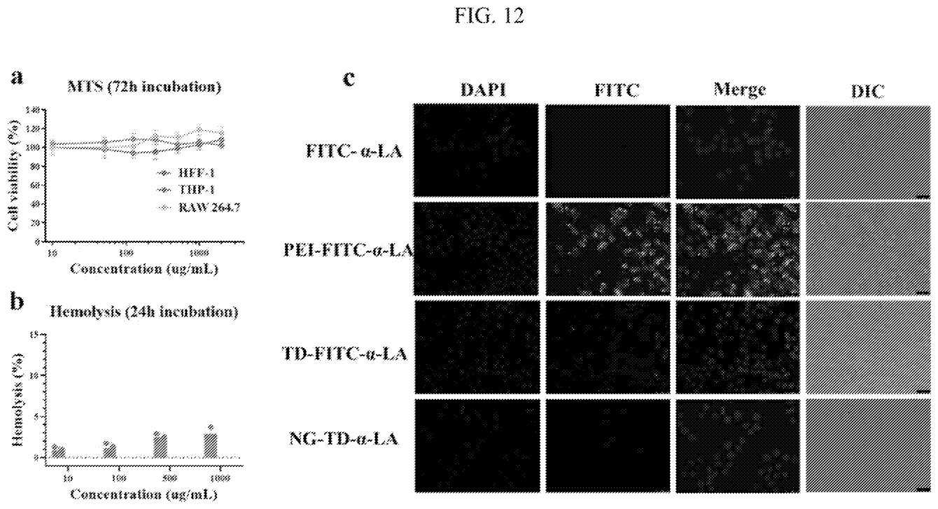

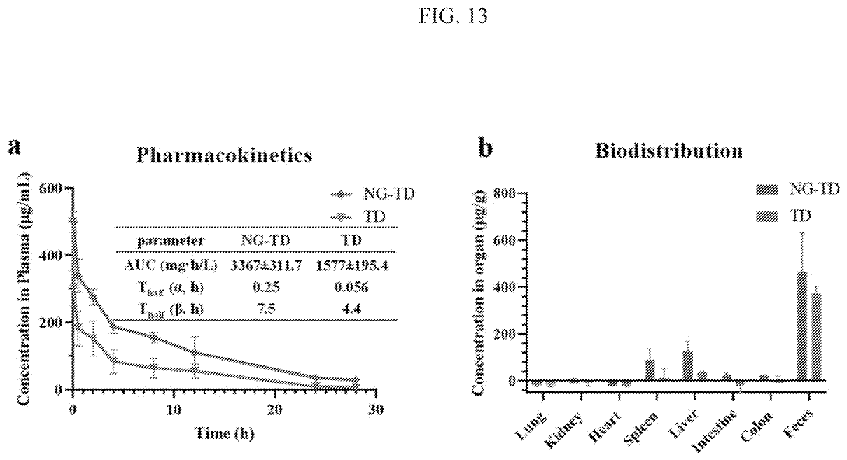

NG-TD toxicity and cellular uptake: The in vitro cytotoxicity of NGs was tested in cell culture and measured by MTS assay. NG-TD shows nontoxic to both immune cell lines (RAW 264.7 and THP-1 cells) normal cell line (HFF-1 cells) and up to 2 mg/mL after 72 h incubation (FIG. 12a). In addition, NGs were incubated with red blood cells in the concentration range of 10-1000 μg/mL for 24 h. The NGs barely show any hemolysis even at highest concentration 1000 μg/mL (FIG. 12b).

The nanotrap is designed to scavenge circulating septic molecules, the phagocytosis of nanogel is preferred to be low to ensure longer circulation and effective immune modulation. It has been demonstrated that nanoparticles with antifouling PEG chain on the surface have long circulation time in human blood with the reduced nonspecific uptake by macrophages. Immune cell uptake was evaluated in cell culture of murine macrophage RAW 264.7 to determine the stealth properties of the NG-TD compare with TD only. The anionic poly(ethylene imine) (PEI) were used as the positive control. NG, TD and PEI were preincubated with FITC-α-LA for 30 min for complete adsorption as shown in agarose gel electrophoresis (FIG. S23a), then added into the cell culture medium (final concentration of α-LA is 5 μg mL-1). The cells were fixed, and nuclei were stained with DAPI after 30 min incubation for microscope observation. As shown in FIG. 12c, the PEI have effective uptake by immune cells, due to the positive charges. The self-assembled TD micelle with Arg groups shows positive surface charge (Table S1), which can be uptake by macrophages. The NG-TD can significantly decrease the cellular uptake due to its neutral surface. The quantitative fluorescent intensity analysis on cell signals normalized by cell numbers confirmed these results (FIG. S23b).

Stability of HNPs in plasma: An effective therapeutic must not only sequester LPS but the NG-LPS complex must also remain soluble in the bloodstream. Aggregation of NG-LPS complexes in the bloodstream could result in lethal side effects by blocking blood flow. The NG-TD (10 mg/mL) with or without overnight incubation with LPS (25:1 mass ratio) in PBS or 50% plasma solution and the size was measured by DLS (FIG. S24). NG kept its size in plasma with or without the addition LPS (˜300 nm). In addition, TEM images of NG-TD after incubation with LPS for overnight in both PBS and plasma indicated the absence of aggregation (no particle size change) (Table S2), indicating that PEG layer of the nanogel inhibits aggregation and NG-LPS complexes are stable in the bloodstream.

TABLE S2
The size of NG or NG/LPS complex
in PBS or in 50% mice plasma.

| | NG in | NG in | NG/LPS | NG/LPS |
|---|---|---|---|---|
| | PBS | plasma | in PBS | in plasma |
| Size(d · nm) | 300.0 ± 18.2 | 292.4 ± 6.4 | 286.2 ± 13.3 | 303.8 ± 15.4 |
| Polydispersity | 0.12 | 0.12 | 0.13 | 0.28 |
| index (PI) | | | | |

Pharmacokinetics and biodistribution of NG: To study the PK profile of NG, we synthesized the Rhodamine B (RB) decorated TD and formed the NG with RB (FIG. S25). The NG (50 mg/kg) was i.v. injected into the tail vein, the TD with equivalent concentration determined by fluorescence (excitation 550 nm, emission 585 nm, microplate reader, BioTek Synergy H1) was injected as comparison. Blood samples were drawn at determined time point for analysis. The TD was rapidly cleared and not detectable beyond 24 h (FIG. 13a). However, NG-TD sustained in blood for longer time, with over 7-fold longer of half-life in comparison with TD and higher area under curve (AUC) (NG-TD 3367±311.7 mg h/L, TD 1577±195.4). After 36 h of injection, the mice were sacrificed, and the major organs were collected and homogenized for the biodistribution study. As shown in FIG. 13b, significantly high concentration of fluorescence was observed in the colon with feces part, which indicated that the NG can scavenging the systemic septic molecules and then excreted through the liver bile-duct.

NG-TD modulate LPS-induced inflammatory response: Intravenous (i.v.) injection of LPS induces septic characteristics such as neutrophil margination and accumulation of proinflammatory cytokines, eventually causing death. Based on the excellent properties of NG-TD (Arg) in vitro, we then further evaluate the LPS and proinflammatory cytokines attenuation capability of nanogel in vivo. Wild type mice were treated with LPS (0.1 mg/mL) via i.v. injection. 5 min later, the mice were treated with either NG-TD (10% PEG^{5k}C17₄Arg₄, 50 mg/mL) or equal volume of PBS (FIG. 14a). Blood was collected from tail vain at 2 h and 4 h after the LPS injection to analysis the plasma cytokine level. TNF-α is the early stage proinflammatory cytokine, which peaked at 1 or 2 h after injection. As shown in FIG. 14b, the PBS treatment group showed high TNF-α level (˜270 μg/mL) in plasma 2 h after LPS injection, which gradually decreased to around 80 μg/mL at 4 h. The nanogel treatment significantly reduced the level of TNF-α at both time points. At 4 h, the nanogel treatment group exhibited similar TNF-α level as normal mice. The IL-6 normally peaked at around 3 or 4 h after stimulation.²⁰ The IL-6 level in PBS treatment group approached to about 8 ng/mL at 2 h and then slightly increased to 10 ng/mL at 4 h. However, the IL-6 can be removed from the bloodstream by nanogel dramatically. Especially at 4 h, the PBS treated group showed a continual increase, however, with the treatment of nanogel, the level can be five times reduced, which even lower than the level at 2 h. These results indicated that the nanogel with long Pk can scavenge circulating LPS and cytokines in the bloodstream and modulate the immune system.

To demonstrate this result, we use the HIV-LTR/Luciferase (HLL) mice as luciferase reporter for NF-κB activation and inflammatory responses to LPS stimulation with/without nanogels. NG/PBS were injected into mice by i.v. 5 min later LPS injection (FIG. 14b). Mice were imaged at t=0 h for baseline and at 2, 6, 24 and 48 h after LPS injection to monitor the bioluminescent signal. As shown in FIG. 14e, LPS-treated mice exhibited significantly increased luminescent signal space at 2 h and 4 h post injection, which is consistent with the cytokines results in FIG. 14b. However, the signal remained low in mice treated with NG solution throughout the observation, indicating the attenuated activity of LPS. Blank nanogel injection did not induce any inflammatory signals similar as PBS injection. The quantitative result of the luminescent signal was displayed in FIG. 14d. LPS treated group showed a peak 2 h after administration and the NG treatment group maintained low level of bioluminescent signal.

Besides, we tested the protection effect of NG to LPS induced infection in vivo. The NG/PBS were i.v. injected follow by the LPS injection (FIG. S26). At 4 h post injection, the NG treatment group show a significant decrease of proinflammatory cytokine, including TNF-α and IL-6. And the pretreated with NG can also inhibit the luminescent signal increase in NF-κB reporter mice compare with LPS treated group. The results indicate that NG have a LPS and proinflammatory cytokine neutralizing capacity in the bloodstream.

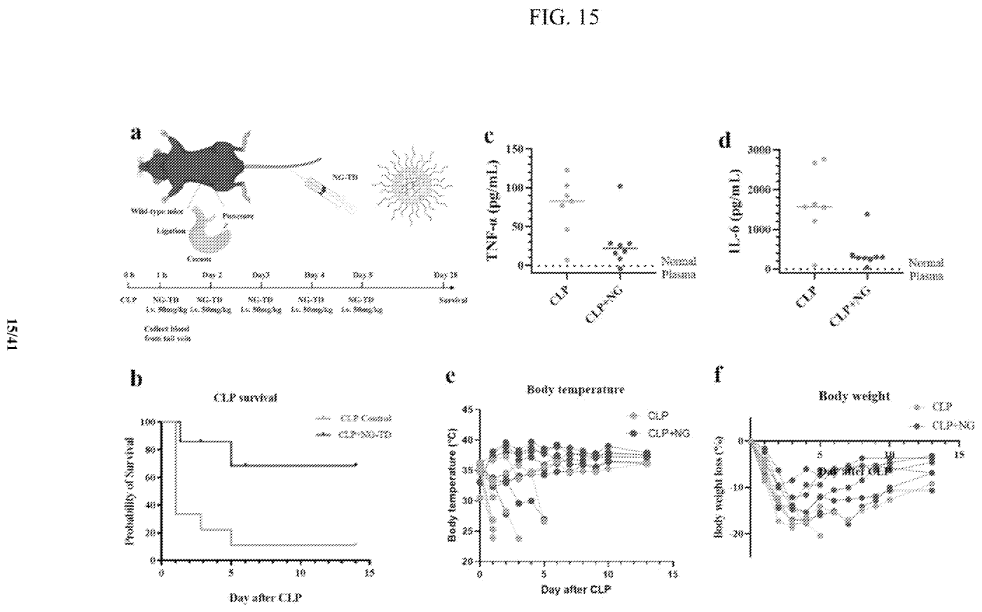

CLP induced polymicrobial Sepsis treatment by NG-TD: Cecal ligation and puncture (CLP), a murine model of bacterial peritonitis, recapitulates key features of secondary bacterial peritonitis in humans, including polymicrobial infection, persistently elevated circulating high mobility group box 1 protein levels, hyperdynamic circulatory system, and the development of acute lung injury is regarded as the clinically equivalent animal model of sepsis.^{21,22} The nanogel was i.v. injected into mice 1 h after the CLP surgery, and followed by four doses from day 2 to day 5 (FIG. 15a). As show in FIG. 15b, CLP surgery induced the severe sepsis and most of the mice dead in 5 days with survival around 11% in 14 days. The NG treatment significantly improved survival of mice from CLP-induced polymicrobial and the survival was around 80%. One day after the CLP surgery, the blood from each group were collected from the tail vain to evaluate the cytokine level by ELISA (FIG. 15c). Compare with the normal mice, the CLP surgery could significantly increase the level of both TNF-α and IL-6, and the mortality events were concentrated in 24 h post CLP, which may cause by hyperinflammatory reaction. The NG treatment can decrease the level of proinflammatory cytokine and prolong the survival, which indicated that the nanogel treatment can capture the secreted cytokines and survive the mice from the cytokine storm. The body temperature and body weight of the survived mice were recorded overtime (FIG. 15e and FIG. 15f). The nanogel treatment can reduce the hypothermia of the CLP mice. The mice showed body weight loss at first few days, then gradually recovered from day 5 in NG treated group.

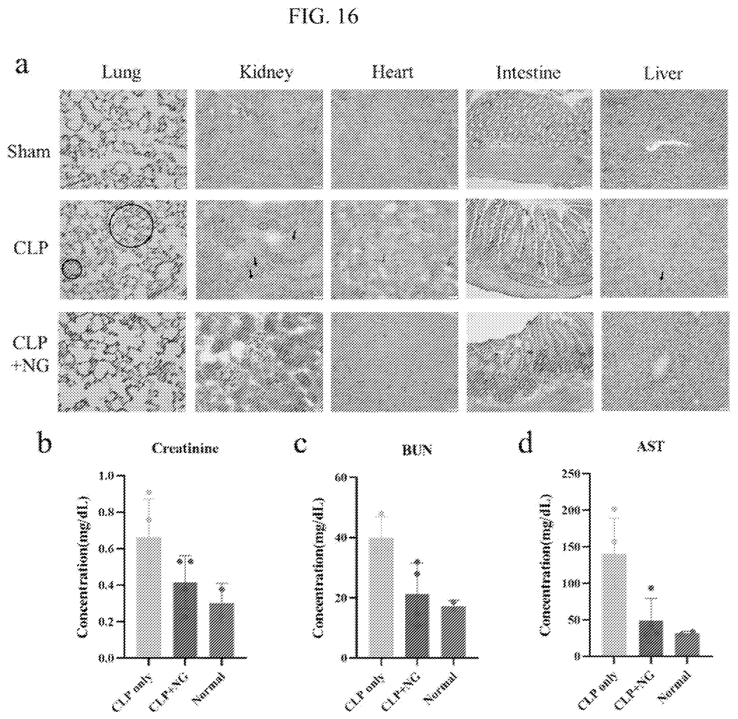

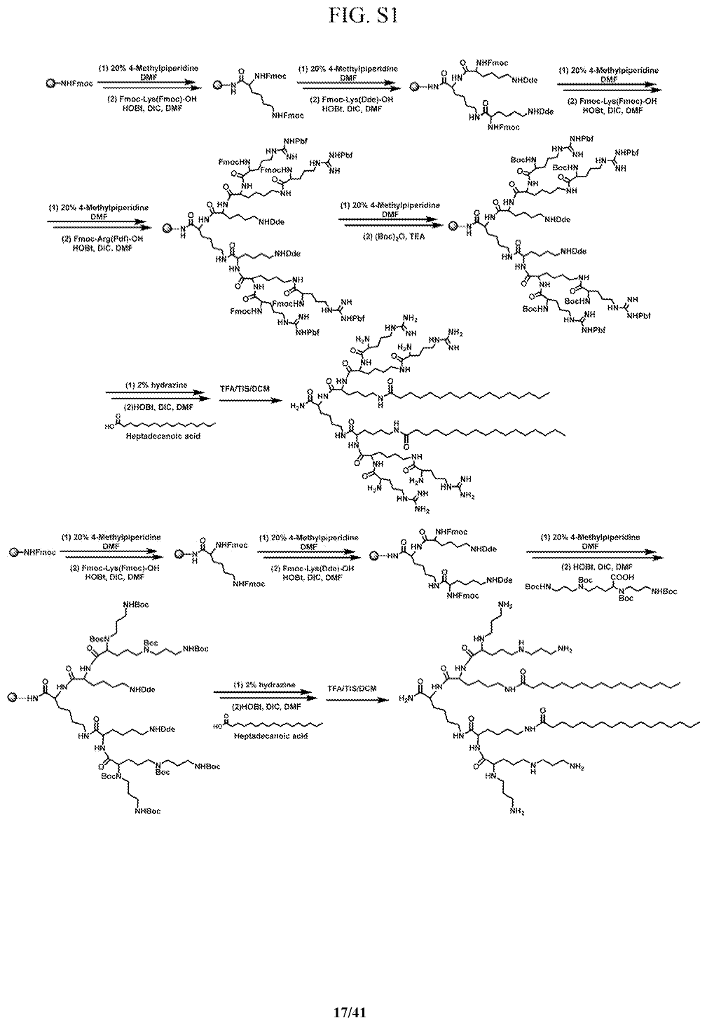

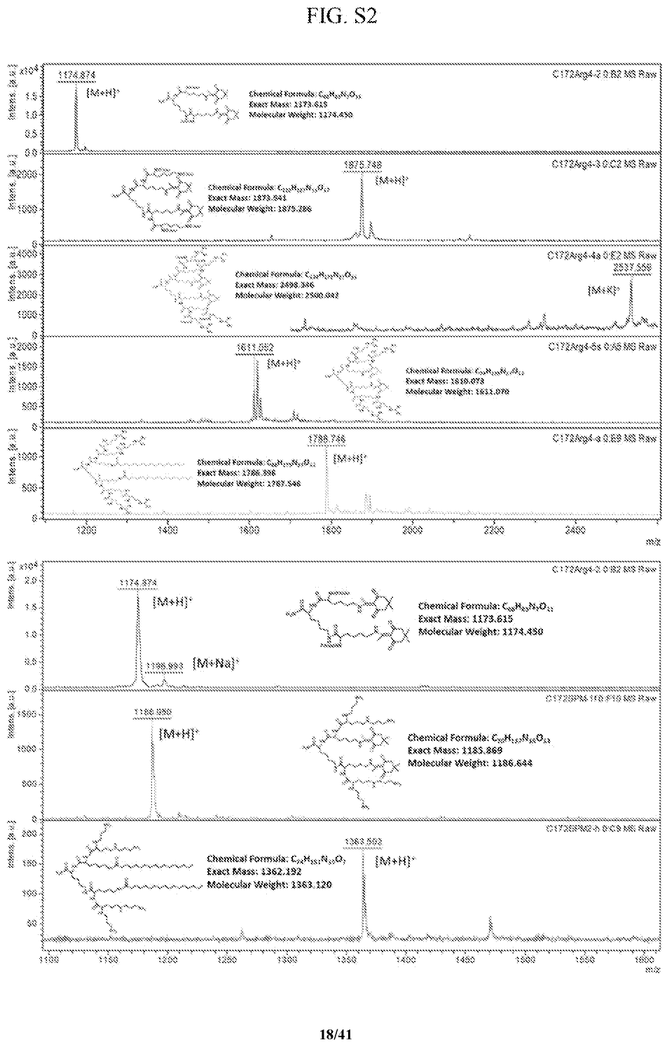

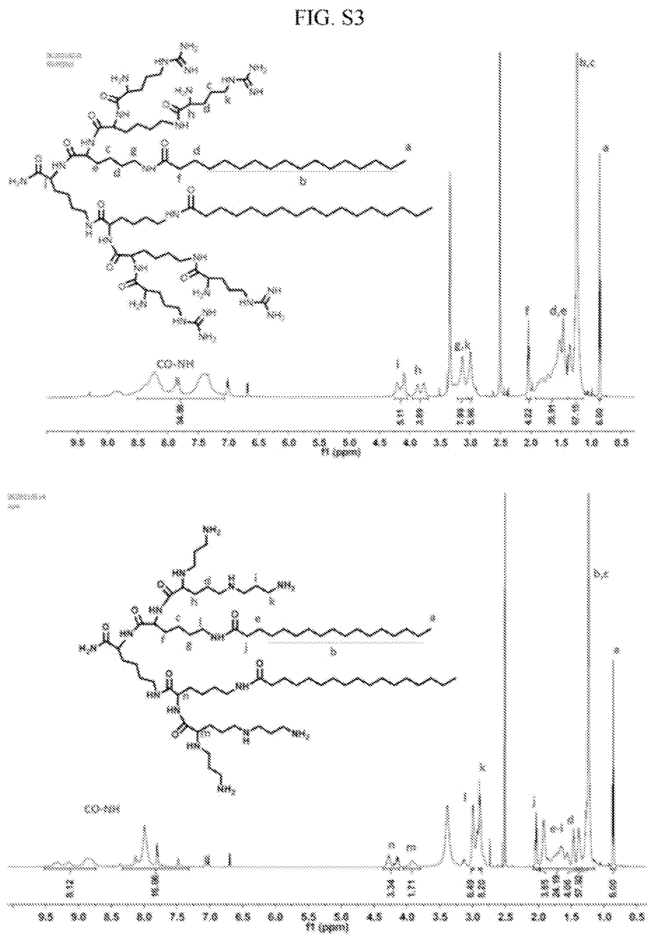

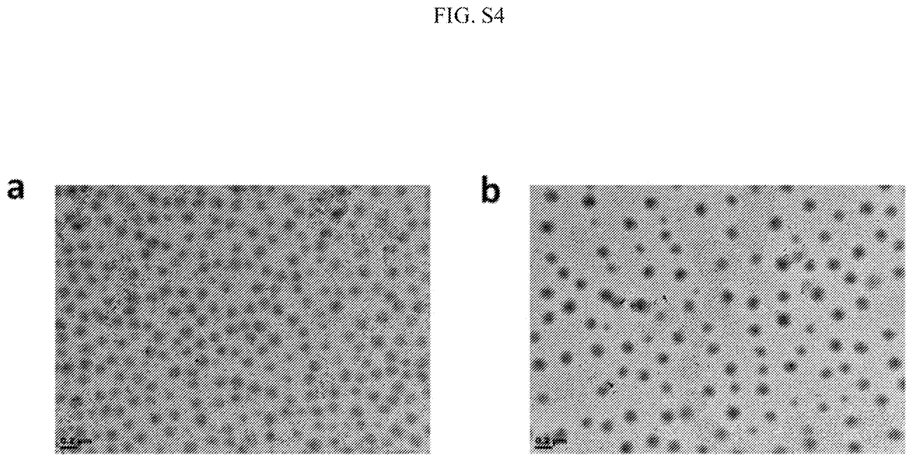

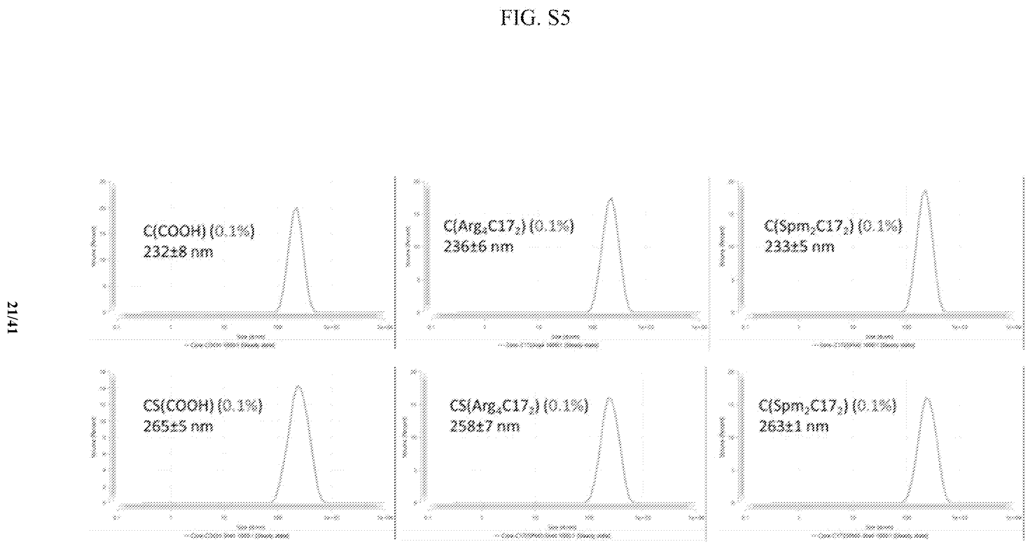

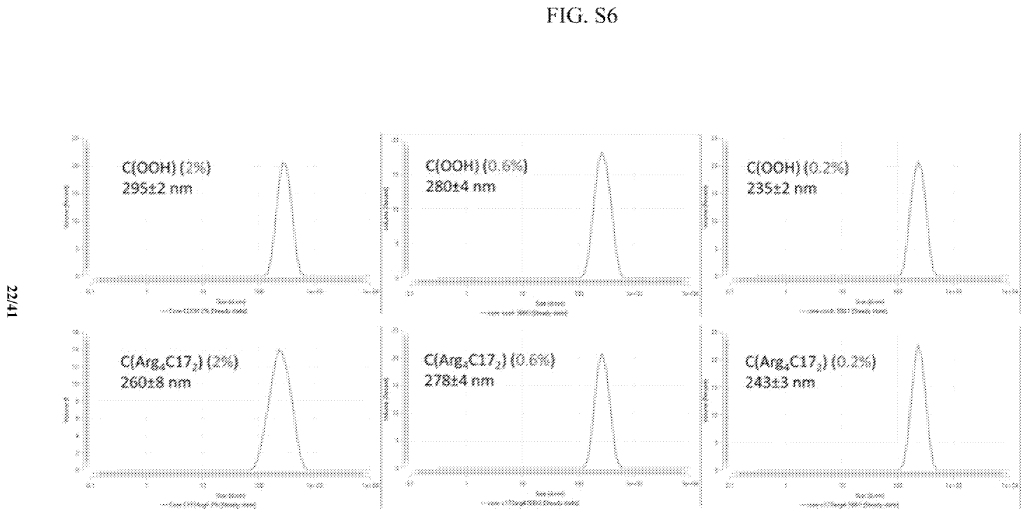

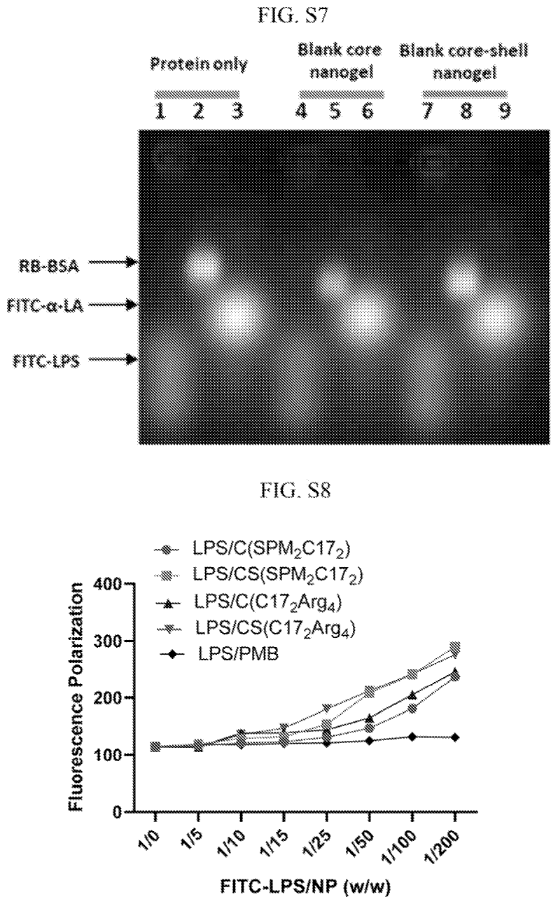

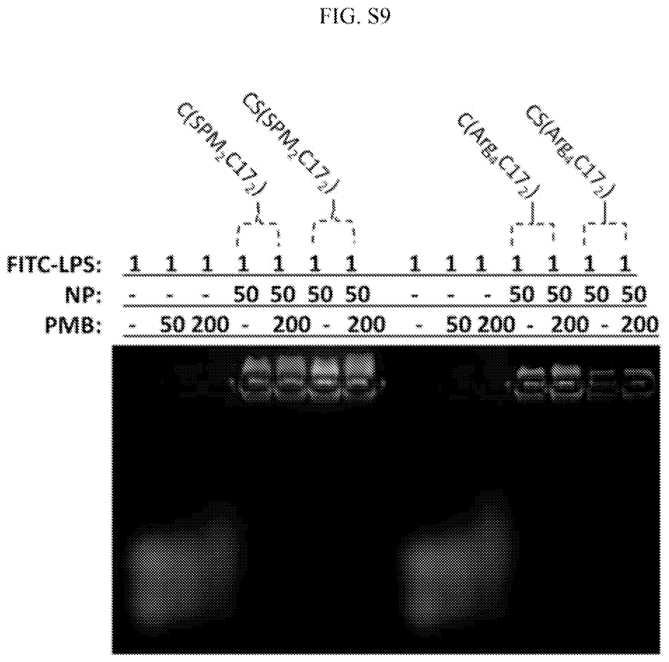

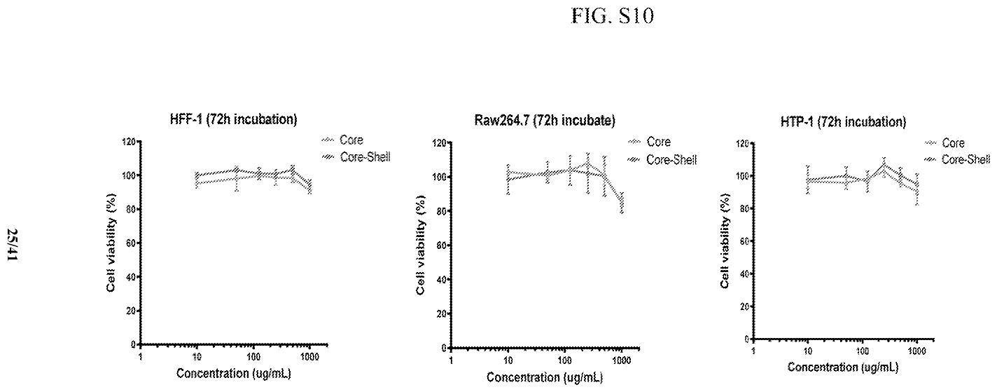

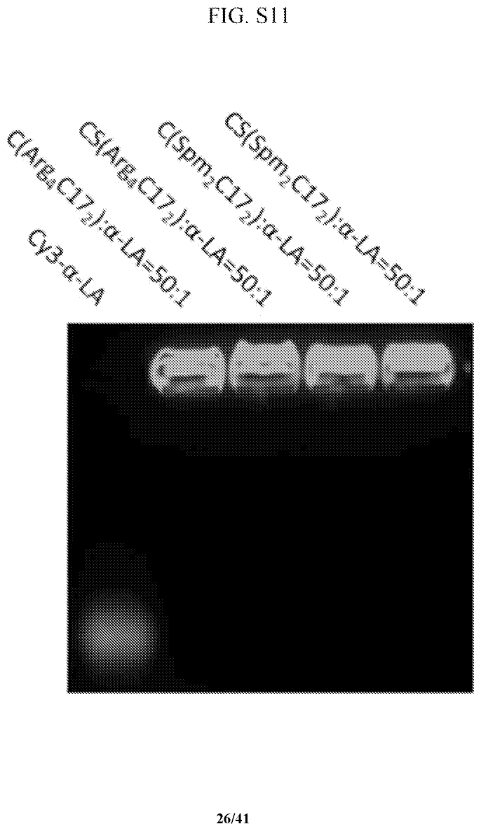

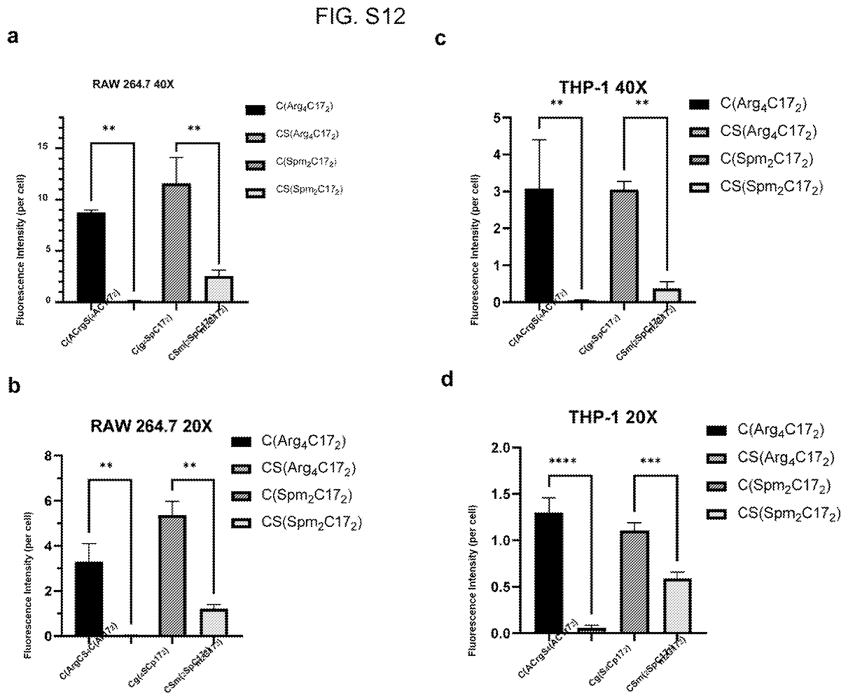

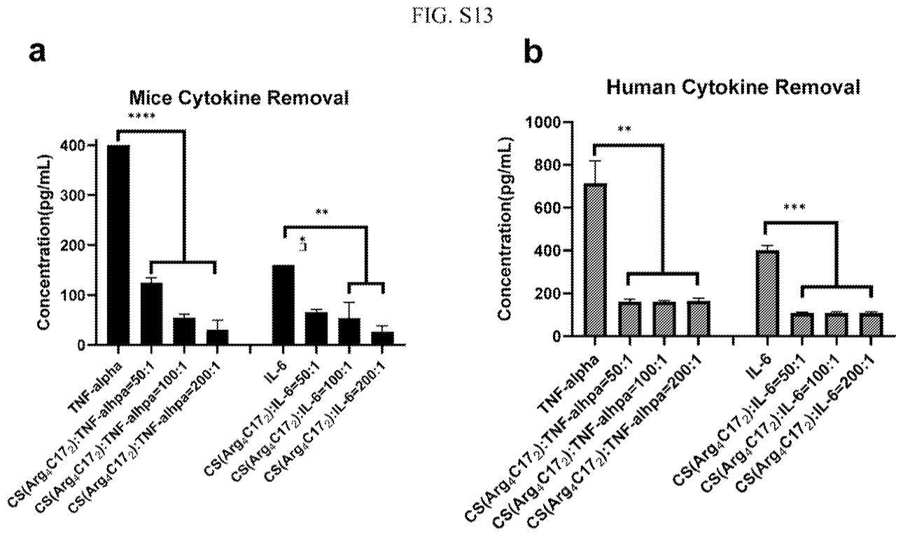

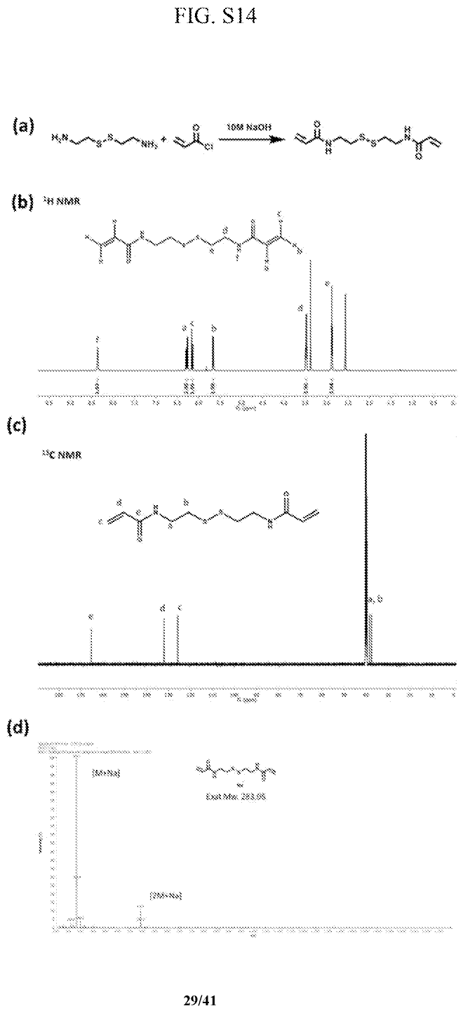

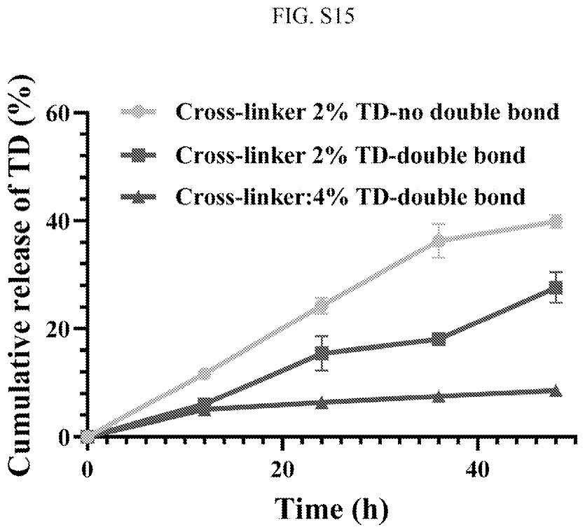

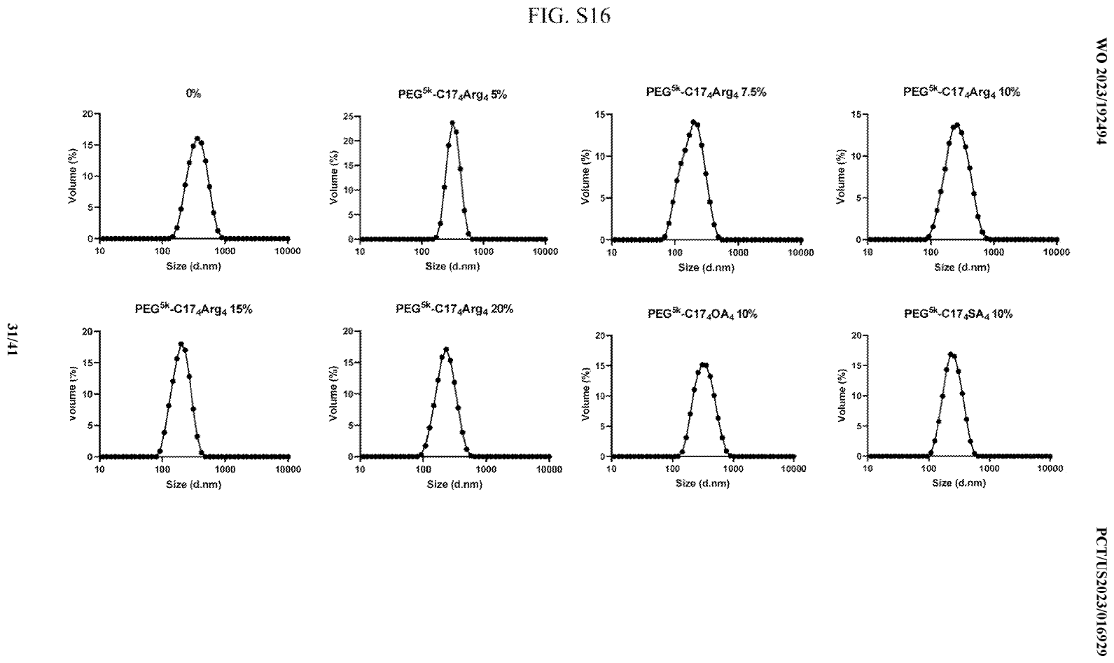

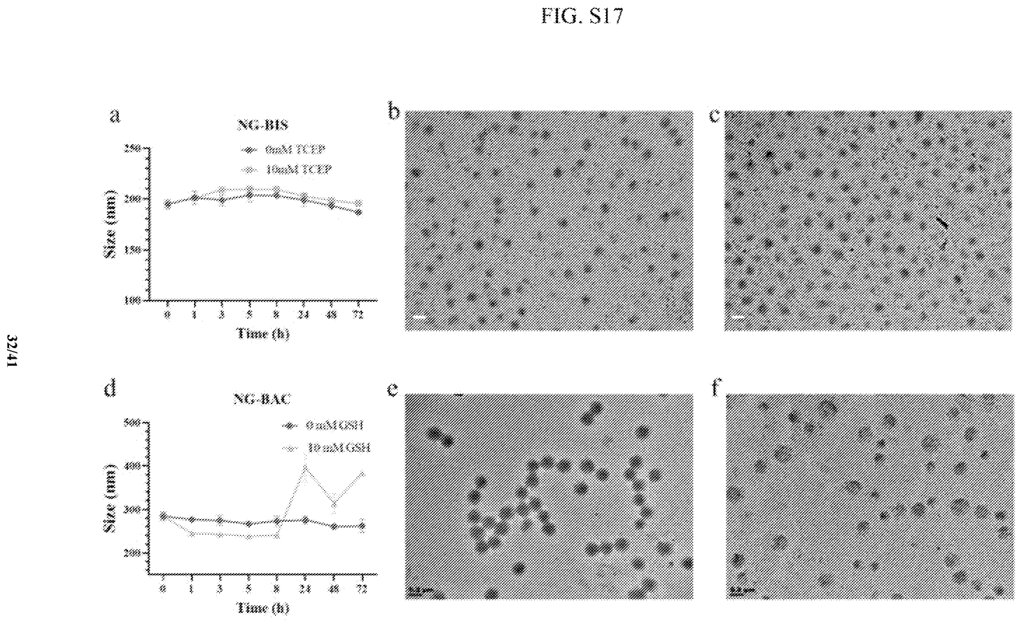

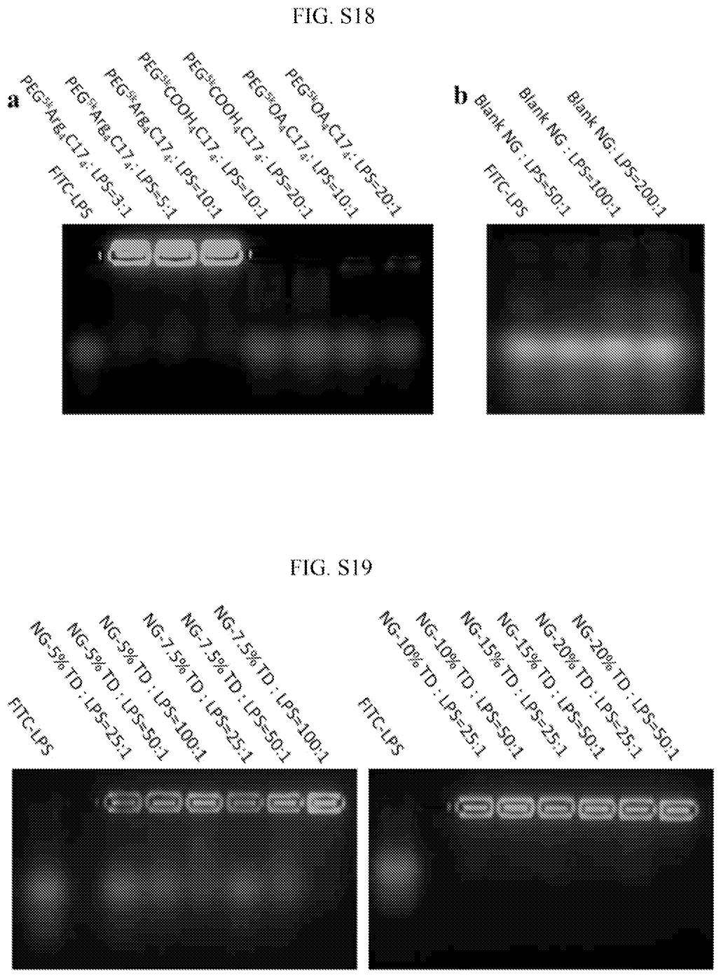

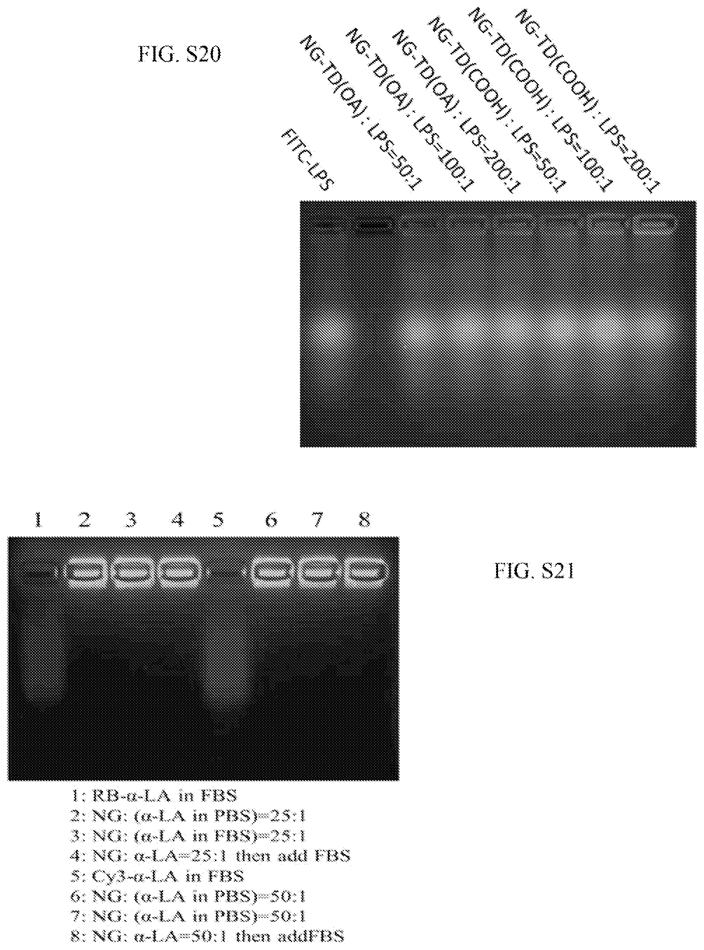

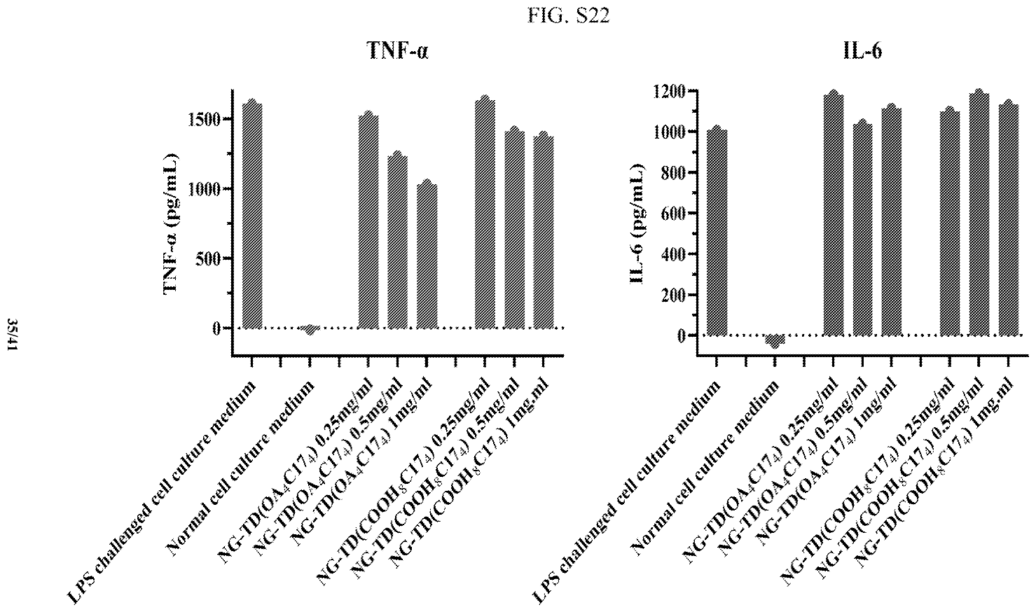

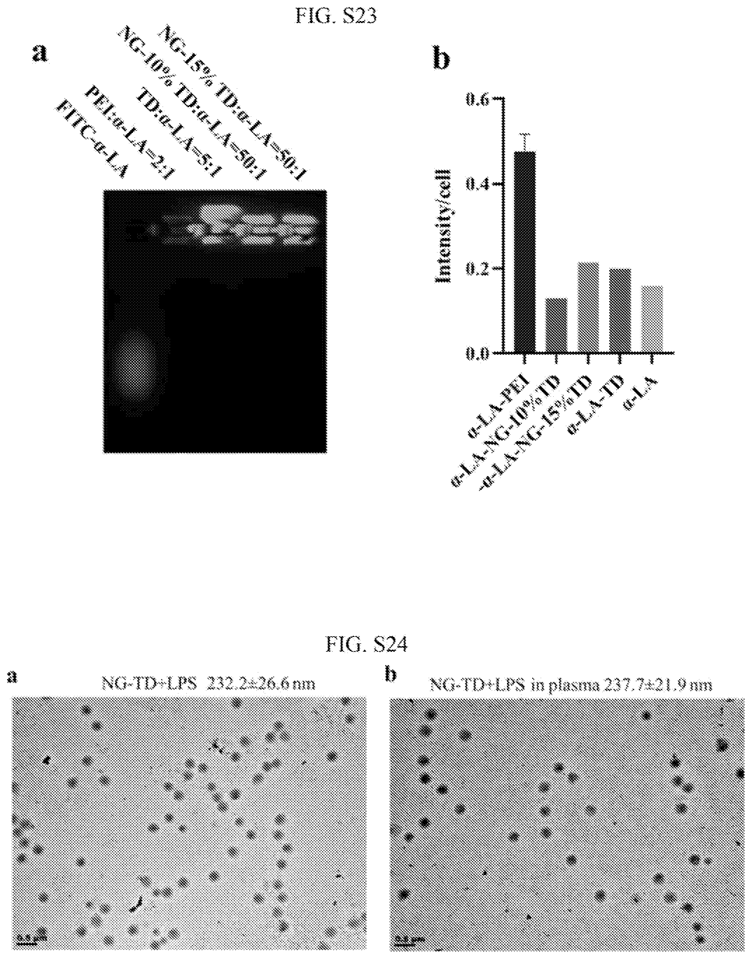

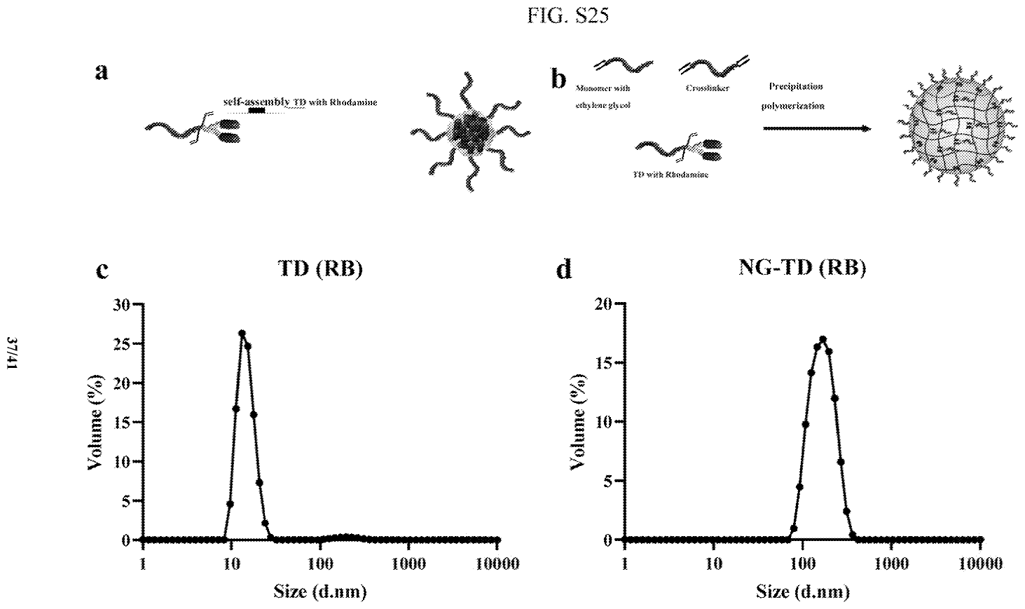

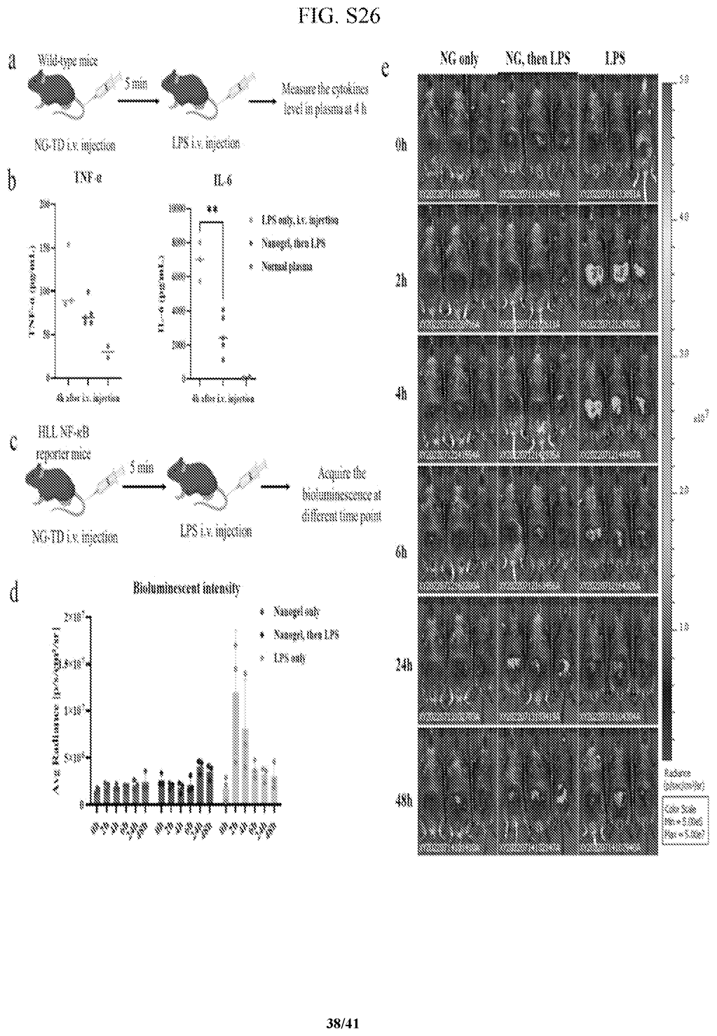

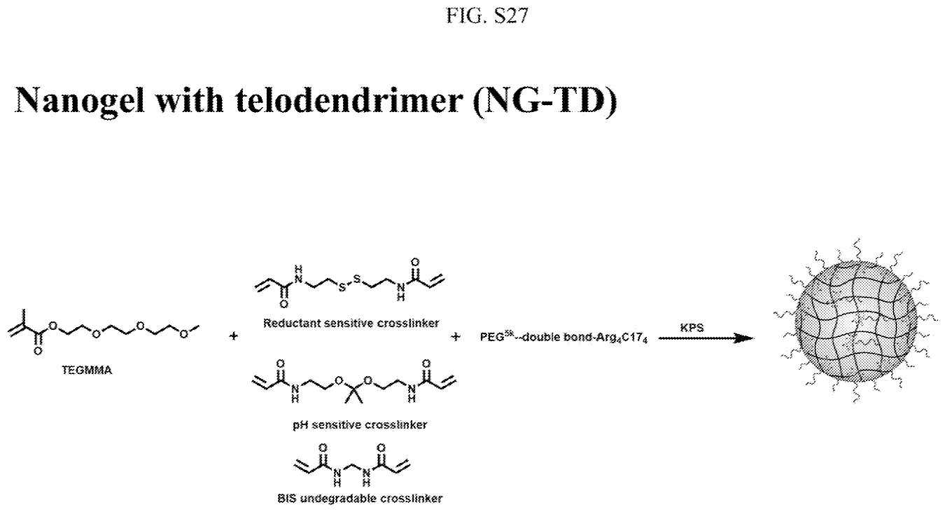

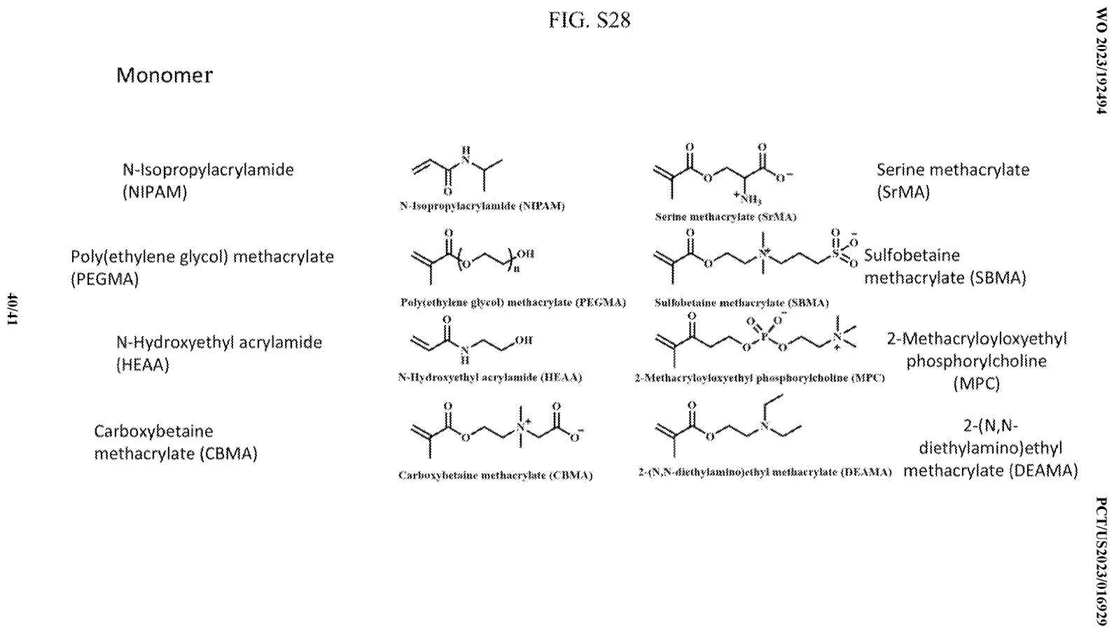

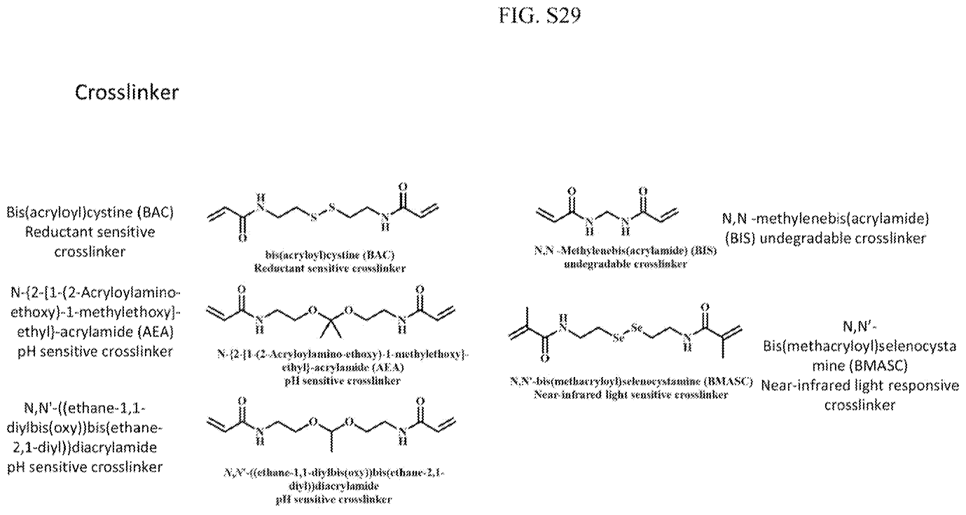

Inspection of H&E sections revealed obvious pathological changes and tissue damages in multiple organs in severe septic mice (FIG. 16a). Significant intra-alveolar hemorrhage with many RBSs (black circle) were observed in CLP mice, which indicated the acute lung injury (ALI). Concurrently, proximal tubular edema and vacuolization (black arrow), tubular epithelial cell simplification (blue circle) were observed in the CLP mice, indicating the tubular injury in the kidney. Besides, in CLP induced sepsis mice, intracellular edema and contraction bands (blue arrow) in cardiomyocytes, foci inflammatory cells infiltration (black arrow) in liver, submucosa necrosis (blue circle) in intestine were observed, indicating multiple organ damage and possible mortality. It was noticed that the NG treatment can improve histopathology in all organs, which showed similar morphology as sham group. Quantification of typical biomarkers associated with kidney (creatinine and BUN) and liver (AST) functions showed no abnormally increased levels for the CLP NG-treated animals in comparison to those of the nontreated group (FIG. 16b-FIG. 16d); notably increased markers reveal liver and kidney injuries.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

1. A biomolecule-binding nanogel composition comprising: a plurality of nanoparticles, each nanoparticle containing a nano-sized crosslinked hydrogel system, the nanoparticle having a spherical surface and within the system a plurality of dendritic functionalized telodendrimer containing a plurality of hydrophobic groups and hydrophilic charged binding moieties, the telodendrimer is bonded to a linear polyethylene glycol polymer (PEG) chain and polymerized in the hydrogel system by at least one double bond located between the linear polyethylene glycol polymer (PEG) chain and the dendritic functionalized telodendrimer, the linear polyethylene glycol polymer (PEG) chain extending on the surface of the nanoparticle to protect a size-exclusive nanogel network.

2. The biomolecule-binding nanogel composition of claim 1, wherein nano-sized crosslinked hydrogel system is polymerized from at least one hydrophilic monomer selected from the group consisting of:

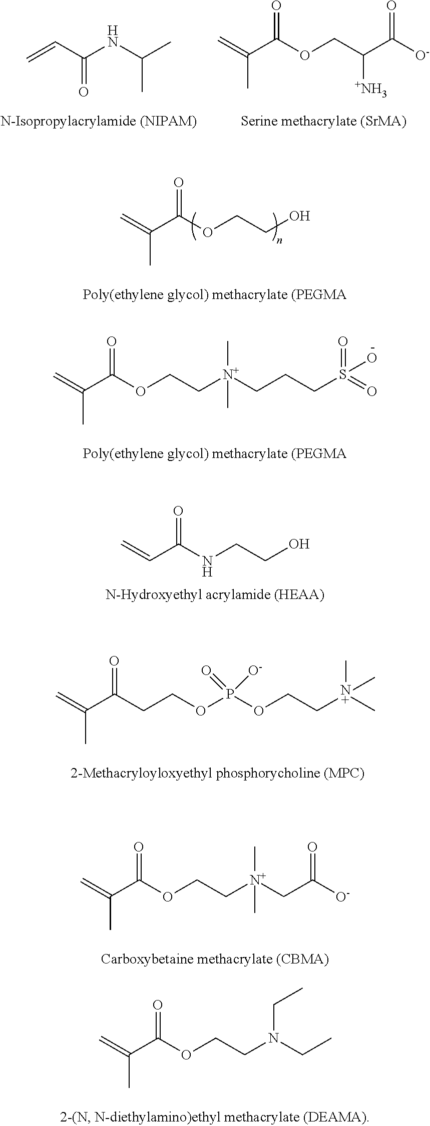

3. The biomolecule-binding nanogel composition of claim 1, wherein nano-sized crosslinked hydrogel system is polymerized from at least one hydrophilic monomer selected from the group consisting of Diethylene glycol methyl ether methacrylate, Triethylene glycol methyl ether methacrylate, Tetraethylene glycol methyl ether methacrylate, and Pentaethylene glycol methyl ether methacrylate.

4. The biomolecule-binding nanogel composition of claim 1, wherein nano-sized crosslinked hydrogel system is polymerized from a biodegradable crosslinking monomer having a crosslinker monomer and a crosslinker molar ratio of about 0.1% to about 20%.

5. The biomolecule-binding nanogel composition of claim 4, wherein at least one biodegradable crosslinker is selected from the group consisting of:

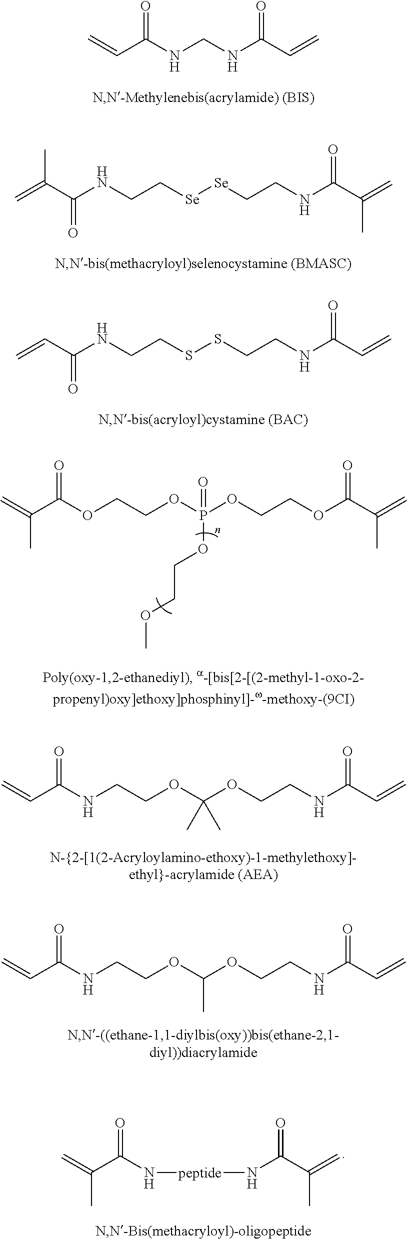

6. The biomolecule-binding nanogel composition of claim 1, wherein the nanoparticles range in size from about 20 nm to about 1000 nm.

7. The biomolecule-binding nanogel composition of claim 1, wherein polyethylene glycol polymer (PEG) chain has molecular weight from about 44 Dalton to about 40,000 Dalton.

8. The biomolecule-binding nanogel composition of claim 1, wherein hydrophobic group is at least one selected from the group consisting of long-chain alkanes (C₁-C₅₀), fatty acids (C₁-C₅₀), aromatic molecules, esters, halogens, nitrocompounds, anthracyclines, fluorocarbons, silicones, steroids, cholesterol, terpenoids, vitamins, and polymers, and amphiphilic groups, cholic acid, riboflavin, and chlorogenic acid.

9. The biomolecule-binding nanogel composition of claim 1, wherein the charged binding moiety is a negatively charged moiety, derivative, or analog of hydroxyl, carboxyl, phosphate, sulfonate, methanesulfonamide, squaric acid, sulfonamide, or oxalic acid, and/or a positively charged moiety, derivative, or analog of arginine, guanidine, amidine, primary amine, secondary amine, tertiary amine, quaternary amine, or tetrazole.

10. The biomolecule-binding nanogel composition of claim 1, wherein the hydrophobic binding group is heptadecanoic acid (C17) and the charged binding moiety is arginine (Arg).

11. The biomolecule-binding nanogel composition of claim 1, wherein the biomolecules that bind to the hydrophobic groups and hydrophilic charged binding moieties include lipopolysaccharides (LPS), small molecule drugs, peptides, proteins, and cytokines.

12. A method of treating uncontrolled systemic inflammatory response in a mammal, said method comprising:
administering to a mammal in need thereof a therapeutically effective amount of a biomolecule-binding nanogel composition comprising, a plurality of nanoparticles, each nanoparticle containing a nano-sized crosslinked hydrogel system, the nanoparticle having a spherical surface and within the system a plurality of dendritic functionalized telodendrimer containing a plurality of hydrophobic groups and hydrophilic charged binding moieties, the telodendrimer is bonded to a linear polyethylene glycol polymer (PEG) chain and polymerized in the hydrogel system by at least one double bond located between the linear polyethylene glycol polymer (PEG) chain and the dendritic functionalized telodendrimer, the linear polyethylene glycol polymer (PEG) chain extending on the surface of the nanoparticle to protect a size-exclusive nanogel network.

13. A pharmaceutical composition comprising the composition of claim 1, wherein the pharmaceutical composition is an injectable formulation.

14. A biomolecule-binding hydrogel composition comprising: a plurality of core-shell nanoparticle, the core-shell nanoparticle having a core and a shell, the core comprises a crosslinked hydrogel system and a plurality of functionalized telodendrimers comprising a plurality of charged and hydrophobic binding moieties, the system having a crosslinking density, the shell comprises a plurality of hydrophilic crosslinked polymers to provide a coating of the entire core, the coating having a crosslinking density that is no less than the crosslinking density of the core.

15. The biomolecule-binding hydrogel composition of claim 14, wherein shell hydrophilic crosslinked telodendrimers are polymerized from at least one hydrophilic monomers selected from the group consisting of Diethylene glycol methyl ether methacrylate, Triethylene glycol methyl ether methacrylate, Tetraethylene glycol methyl ether methacrylate, Pentaethylene glycol methyl ether methacrylate, N-Isopropylacrylamide (NIPAM), acrylic acid (AA), N-Hydroxyethyl acrylamide (HEAA), Carboxybetaine methacrylate (CBMA), Serine methacrylate (SrMA), Sulfobetaine methacrylate (SBMA), 2-Methacryloyloxyethyl phosphorylcholine (MPC), and 2-(N,N-diethylamino)ethyl methacrylate (DEAMA).

16. The biomolecule-binding hydrogel composition of claim 14, wherein core functionalized crosslinked telodendrimers are polymerized from at least one hydrophilic monomer selected from the group consisting of Diethylene glycol methyl ether methacrylate, Triethylene glycol methyl ether methacrylate, Tetraethylene glycol methyl ether methacrylate, Pentaethylene glycol methyl ether methacrylate.

17. The biomolecule-binding hydrogel composition of claim 14, wherein core functionalized crosslinked telodendrimers are polymerized from at least one hydrophilic monomer selected from the group consisting of:

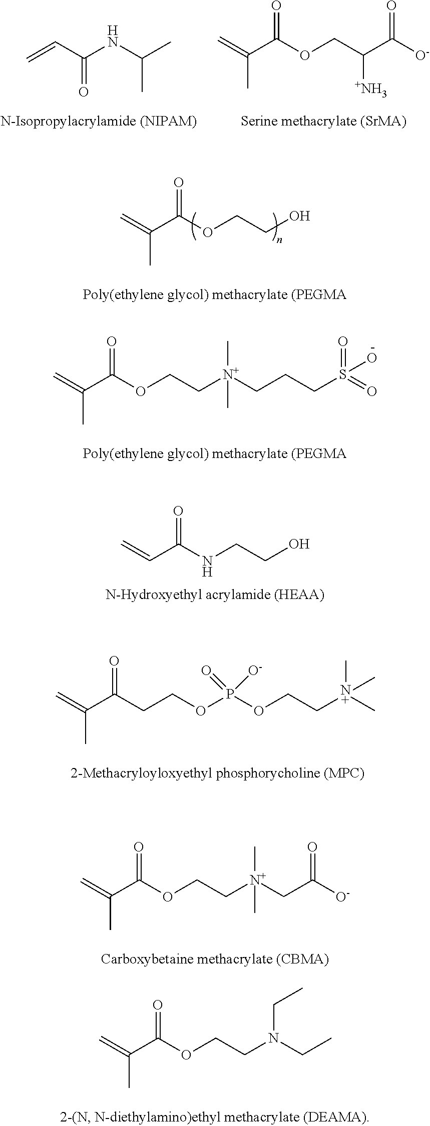

18. The biomolecule-binding hydrogel composition of claim 14, wherein core contains from about 0.1% to about 20% acrylic acid for functionalized telodendrimer conjugation.

19. The biomolecule-binding hydrogel composition of claim 14, wherein at least one biodegradable crosslinker is selected from the group consisting of:

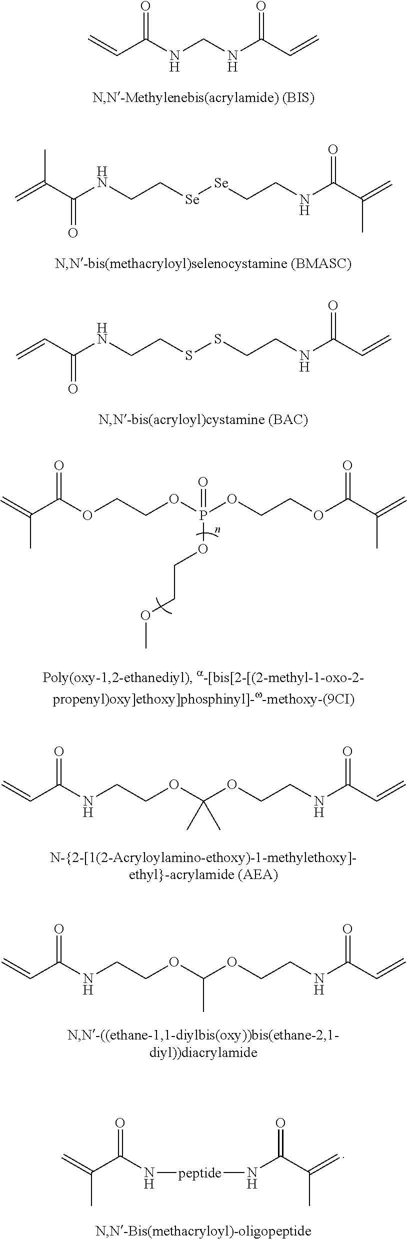

20. The biomolecule-binding hydrogel composition of claim 14, wherein binding hydrophobic moiety is at least one selected from the group consisting of long-chain alkanes (C₁-C₅₀), fatty acids (C₁-C₅₀), aromatic molecules, esters, halogens, nitrocompounds, anthracyclines, fluorocarbons, silicones, certain steroids such as cholesterol, terpenoids, vitamins, and polymers, and amphiphilic groups, cholic acid, riboflavin, and chlorogenic acid, wherein the charged binding moiety is a negatively charged moiety, derivative, or analog of hydroxyl, carboxyl, phosphate, sulfonate, methanesulfonamide, squaric acid, sulfonamide, or oxalic acid, and/or a positively charged moiety, derivatives, or analog of arginine, quanidine, amidine, primary amine, secondary amine, tertiary amine, quaternary amine, or tetrazole, wherein the hydrophobic moiety is heptadecanoic acid (C17) and the charged moieties are arginine (Arg) and/or spermine (Spm), and wherein shell hydrophilic crosslinked hydrogel are polymerized from poly(N-isopropylacrylamide-co-2-methacryloyloxyethyl phosphorylcholine) (poly(NIPAm-co-MPC)).

21-28. (canceled)
